SEQUENCE LISTING (2 sequences)

SEQ ID NO: 1 - protein, 505 aa, unidentified
  source 1..505: note = human papillomavirus
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPTDLYIKGSGSTATLANSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 2 - protein, 473 aa, unidentified
  source 1..473: note = human papillomavirus
MRHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

(19) United States
(12) Patent Application Publication
(10) Pub. No.: US 2026/0102498 A1
(43) Pub. Date: Apr. 16, 2026

(54) METHODS AND COMPOSITIONS FOR PRODUCING A THERAPEUTIC FIELD EFFECT

(71) Applicant: Aura Biosciences, Inc., Boston, MA (US)

(72) Inventors: Joseph McQuaid, Boston, MA (US); Elisabet de los Pinos, Boston, MA (US); Keith Faucher, Boston, MA (US)

(73) Assignee: Aura Biosciences, Inc., Boston, MA (US)

(21) Appl. No.: 19/359,229

(22) Filed: Oct. 15, 2025

Related U.S. Application Data
(60) Provisional application No. 63/708,172, filed on Oct. 16, 2024.
(60) Provisional application No. 63/724,011, filed on Nov. 22, 2024.
(60) Provisional application No. 63/743,688, filed on Jan. 10, 2025.
(60) Provisional application No. 63/774,561, filed on Mar. 19, 2025.
(60) Provisional application No. 63/794,556, filed on Apr. 25, 2025.

(51) Int. Cl. A61K 41/00; A61K 9/00; A61K 35/76; A61K 47/69; A61P 35/00

(52) U.S. Cl. CPC A61K 41/0071; A61K 9/0019; A61K 35/76; A61K 41/0061; A61K 47/6901; A61P 35/00

(57) ABSTRACT

The disclosure relates to methods and composition for treating a target tumor by delivering to the base of the target tumor a pharmaceutical composition comprising a virus-related particle in a therapeutically effective amount sufficient to (i) treat the target tumor and (ii) induce a therapeutic field effect and/or transform an immunologically inactive tumor into an immunologically active tumor.

### RELATED APPLICATIONS

This application claims the benefit under 35 U.S.C. § 119 (e) of U.S. provisional application No. 63/708,172, filed Oct. 16, 2024, U.S. provisional application No. 63/724,011, filed Nov. 22, 2024, U.S. provisional application No. 63/743,688, filed Jan. 10, 2025, U.S. provisional application No. 63/774,561, filed Mar. 19, 2025, and U.S. provisional application No. 63/794,556, filed Apr. 25, 2025, each of which is incorporated by reference herein in its entirety

### REFERENCE TO AN ELECTRONIC SEQUENCE LISTING

The contents of the electronic sequence listing (A091570023US05-SEQ-HJD.xml; Size: 4,084 bytes; and Date of Creation: Oct. 14, 2025) are herein incorporated by reference in their entirety.

### BACKGROUND

Following heart disease, cancer is the leading cause of death in the United States and the primary cause of death worldwide. Incidents and mortality rates continue to increase, and an estimated 1.9 million newly diagnosed cases and predicted 600,000 deaths are expected in 2021. In particular, there is a large increase in the number of early-stage cancers requiring treatment options with improved benefit-risk profile. Of these cases, most are solid tumors with bladder being the 9th most common cancer occurring globally and sixth most common in the United States. Approximately 70-80% of all patients diagnosed with bladder cancer initially present as a heterogeneous group of superficial tumors known as NMIBC. Although NMIBC has a relatively high rate of survival over five years (>88%), approximately 70% of cases recur after initial treatment with numbers as high as 20% progressing to muscle invasive disease.

### SUMMARY

There is a high unmet medical need for function-preserving organ-sparing therapies for the treatment of early stage, local cancers. One of the most prevalent cancers that present with local disease (non-metastatic) at the time of diagnosis is urothelial cancer, including urothelial cancer of the bladder (UCB). Provided herein, in some aspects, are methods for treating urothelial cancer, including UCB, that unexpectedly result in a complete clinical response with a bladder urothelial field effect, even in the absence of combination therapy with an immune checkpoint inhibitor. This method, in some instances, includes (but is not limited to) a focal injection (e.g., a single, low dose injection) of a pharmaceutical composition comprising a virus-like particle conjugated to a light-activatable drug payload (e.g., a virus-like drug conjugate), with light activation. This complete clinical response includes direct tumor cell necrosis and pro-immunogenic cell death that elicits a robust and durable anti-tumor immune response, while preserving organ function. The Phase 1 clinical data described herein demonstrates that these responses were achieved in patients with non-muscle-invasive bladder cancer (NMIBC) without systemic exposure of the virus-like drug conjugate, without the use of an immune checkpoint inhibitor, and without serious adverse effects often observed with conventional bladder cancer treatments. Clinical data showed histopathological evidence of robust immune activation, with a well-tolerated safety profile in all patients receiving the treatments provided herein—and importantly, bladder function in all patients was preserved.

In some instances, treatment with a virus-like drug conjugate resulted in transformation of an immunologically “silent” tumor—one that lacks T-cell infiltration and exhibits poor antigen presentation—into an inflamed, immune-active environment capable of responding to immunotherapy, for example. As described herein, injection of a virus-like drug conjugate directly into or proximate to a primary target tumor induces local inflammation within the tumor microenvironment. This leads to release of tumor-associated antigens, activation of dendritic cells, and recruitment and priming of cytotoxic CD8⁺ T cells, among other immune cell types. The resulting cascade enhances antigen presentation and promotes immune cell infiltration, thereby converting the tumor from “cold” to “hot.” Such conversion can sensitize previously resistant tumors to immune checkpoint blockade and other immunotherapeutic approaches.

Aspects of the technology relate to a method for treating a target tumor in a subject in need thereof, the method comprising: injecting into the base of a target tumor and/or into a site proximate to the base of the target tumor a pharmaceutical composition comprising a therapeutic agent in a therapeutically effective amount sufficient to treat the target tumor and induce a therapeutic field effect.

Other aspects of the technology relate to a method for treating a target tumor in a subject in need thereof, the method comprising: injecting into the base of a target tumor and/or into a site proximate to the base of the target tumor a pharmaceutical composition comprising a therapeutic agent in a therapeutically effective amount sufficient to transform a cold (immunologically inactive) tumor into a hot (immunologically active) tumor.

In some embodiments, the therapeutic agent is selected from virus-related particles. In some embodiments, the virus-related particles are selected from viruses, virus-like particles, defective viral particles, and pseudoviruses. In some embodiments, the therapeutic agent comprises virus-like particles. In some embodiments, the therapeutic agent comprises a virus-like drug conjugate.

In some embodiments, the therapeutic agent is selected from antibodies and other antigen-binding agents. In some embodiments, the therapeutic agent comprises an antibody-drug conjugate.

In some embodiments, the therapeutic agent is an immunomodulatory agent.

In some embodiments, the pharmaceutical composition further comprises a light-activated moiety. In some embodiments, the therapeutic agent comprises the light-activated moiety. In some embodiments, the light-activated moiety comprises a photosensitizer. In some embodiments, the photosensitizer is selected from porphyrins, aminolevulinic acid, and phthalocyanines.

In some embodiments, the photosensitizer comprises a compound of Formula I:

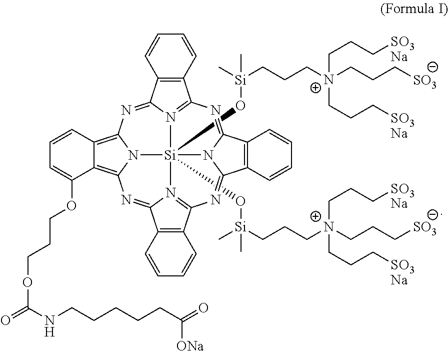

In some embodiments, the photosensitizer comprises a compound of Formula II:

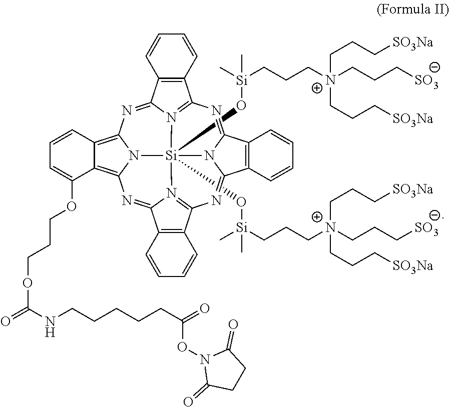

In some embodiments, the target tumor is a target urothelial tumor, and the therapeutic field effect is a therapeutic urothelial field effect. In some embodiments, the target urothelial tumor is a target bladder urothelial tumor, and the therapeutic field effect is a therapeutic bladder urothelial field effect.

In some embodiments, the target bladder urothelial tumor is a non-muscle invasive bladder cancer (NMIBC).

In some embodiments, the therapeutic field effect is without systemic exposure to the therapeutic agent. In some embodiments, the therapeutic field effect is without local or system exposure of an immune checkpoint inhibitor.

In some embodiments, the therapeutic field effect is within 7-14 days of injecting the pharmaceutical composition.

In some embodiments, the pharmaceutical composition is delivered to the bladder wall below the base of the target tumor and above the muscularis propria.

In some embodiments, injecting the pharmaceutical composition is via a single injection or multiple injections (e.g., at least two injections).

In some embodiments, the method further comprises exposing the target tumor to a laser (i.e., a light wavelength of a laser). In some embodiments, the cumulative fluence of exposure of the target tumor to the laser is about 50 J/cm² to about 200 J/cm², optionally about 75 J/cm² or about 100 J/cm².

In some embodiments, the target tumor is exposed to a first light wavelength to visualize the distribution of the therapeutic agent and the target tumor for injection, then exposed to a second light wavelength to activate the light-activated moiety, optionally wherein the cumulative fluence of exposure of the target tumor to the second laser is about 50 J/cm² to about 200 J/cm², optionally about 75 J/cm² or about 150 J/cm².

In some embodiments, the therapeutically effective amount is a 200 μg, 400 ug, or 800 ug dose of the therapeutic agent.

In some embodiments, the method comprises administering a single 200 μg, 400 ug, or 800 ug dose of the therapeutic agent via one or more injection into the base of a target tumor and/or into a site proximate to the base of the target tumor, or administering two or more 200 μg, 400 ug, or 800 ug doses of the therapeutic agent doses via two or more injections into the base of a target tumor and/or into a site proximate to the base of the target tumor.

In some embodiments, the method comprises administering three or more 200 μg, 400 ug, or 800 ug doses of the therapeutic agent doses via three or more injections into the base of a target tumor and/or into a site proximate to the base of the target tumor

In some embodiments, the time between any two doses is about 7 to 21 days, optionally about 14 days.

In some embodiments, the therapeutically effective amount is administered weekly for at least 2 or at least 3 weeks, optionally for about 2 or about 3 weeks.

In some embodiments, exposing the target tumor to a laser occurs 24 hours after the dose of the therapeutic agent is administered.

In some embodiments, a target bladder urothelial tumor is intermediate risk NMIBC. In some embodiments, a subject undergoes a transurethral resection of bladder tumor (TURBT) 26 to 29 days, optionally about 27 days, after administering the dose of the therapeutic agent.

In some embodiments, a target bladder urothelial tumor is intermediate risk NMIBC or high risk NMIBC.

In some embodiments, a target bladder urothelial tumor is intermediate muscle invasive bladder cancer (MIBC) or high risk MIBC.

In some embodiments, a method comprises administering two 200 ug doses of the therapeutic agent via two or more injections into the base of a target tumor and/or into a site proximate to the base of the target tumor, wherein the time between the two doses is 14 days, and wherein the target bladder urothelial tumor is intermediate risk NMIBC.

In some embodiments, a method comprises administering two 400 ug doses of the therapeutic agent doses via two or more injections into the base of a target tumor and/or into a site proximate to the base of the target tumor, wherein the time between the two doses is 14 days, and wherein the target bladder urothelial tumor is intermediate risk NMIBC.

In some embodiments, a method comprises administering two 400 ug doses of the therapeutic agent doses via two or more injections into the base of a target tumor and/or into a site proximate to the base of the target tumor, wherein the time between the two doses is 14 days, wherein the subject undergoes the TURBT about 14 days after the first does is administered, and wherein the target bladder urothelial tumor is intermediate risk NMIBC.

In some embodiments, a method comprises administering two 400 ug doses of the therapeutic agent doses via two or more injections into the base of a target tumor and/or into a site proximate to the base of the target tumor, wherein the time between the two doses is 14 days, wherein the subject undergoes the TURBT about 14 days after the first dose is administered, and wherein the target bladder urothelial tumor is high risk NMIBC.

In some embodiments, a method comprises administering two 800 ug doses of the therapeutic agent doses via two or more injections into the base of a target tumor and/or into a site proximate to the base of the target tumor, wherein the time between the two doses is 14 days, and wherein the target bladder urothelial tumor is intermediate risk NMIBC.

In some embodiments, a method comprises administering two 800 ug doses of the therapeutic agent doses via two or more injections into the base of a target tumor and/or into a site proximate to the base of the target tumor, wherein the time between the two doses is 14 days, wherein the subject undergoes the TURBT about 14 days after the first dose is administered, and wherein the target bladder urothelial tumor is high risk NMIBC.

Some aspects of the technology relate to a bladder cancer therapy, comprising: (a) injecting a pharmaceutical composition comprising a photosensitive virus-like drug conjugate (VDC) into one or more bladder cancer lesions of a patient, wherein the pharmaceutical composition is injected at a dose of about 200 μg, about 400 ug, or about 800 ug of the VDC per bladder cancer lesion; (b) exposing the one or more bladder cancer lesions to a laser treatment at about 24 hours after injecting the pharmaceutical composition into the one or more bladder cancer lesions; and (c) repeating steps (a) and (b).

Some aspects of the technology relate to a bladder cancer therapy, comprising: (a) performing a first cycle of therapy comprising (i) injecting a pharmaceutical composition comprising a photosensitive VDC into one or more bladder cancer lesions of a patient, wherein the pharmaceutical composition is injected at a dose of about 200 μg, about 400 ug, or about 800 ug of the VDC per bladder cancer lesion, and (ii) exposing the one or more bladder cancer lesions to a first laser treatment at about 24 hours after injecting the pharmaceutical composition into the one or more bladder cancer lesions; and (b) performing a second cycle of therapy comprising (i) injecting the pharmaceutical composition into the one or more bladder cancer lesions of the patient, wherein the VDC is injected at a dose of about 200 μg, about 400 ug, or about 800 μg per bladder cancer lesion, and (ii) exposing the one or more bladder cancer lesions to a second laser treatment at about 24 hours after injecting the pharmaceutical composition into the one or more bladder cancer lesions, wherein the second cycle of therapy is performed about 24 hours after the first cycle of therapy is performed.

In some embodiments, a patient is an intermediate risk patient.

In some embodiments, a patient is a high risk patient.

In some embodiments, a therapy further comprising performing TURBT.

In some embodiments, a TURBT is performed about 2 weeks after completing the second cycle of therapy.

In some embodiments, a patient is an intermediate risk patient, and the dose is about 200 ug of the VDC per bladder cancer lesion.

In some embodiments, a patient is an intermediate risk patient, and the dose is about 400 ug of the VDC per bladder cancer lesion.

In some embodiments, a patient is an intermediate risk patient, the dose is about 400 ug of the VDC per bladder cancer lesion, and the therapy further comprises performing TURBT about 2 weeks after completing the second cycle of therapy.

In some embodiments, a patient is a high risk patient, the dose is about 400 ug of the VDC per bladder cancer lesion, and the therapy further comprises performing TURBT about 2 weeks after completing the second cycle of therapy.

In some embodiments, a patient is an intermediate risk patient, and the dose is about 800 ug of the VDC per bladder cancer lesion.

In some embodiments, a patient is a high risk patient, the dose is about 800 ug of the VDC per bladder cancer lesion, and the therapy further comprises performing TURBT about 2 weeks after completing the second cycle of therapy.

In some embodiments, a pharmaceutical composition is injected using a needle having a 2-4 mm tip. In some embodiments, the needle is a Laborie injeTAK® needle.

In some embodiments, about 1 ml of the pharmaceutical composition is injected into the target tumor, wherein the therapeutically effective amount delivered is about 200 μg to about 800 ug.

In some embodiments, exposing the target tumor to a laser comprises: (a) advancing a cystoscope comprising a treatment laser into the bladder and positioning the treatment laser over the target tumor midpoint to produce a laser spot size of about 2 cm; and (b) delivering a total light dose of about 100 J/cm². In some embodiments, a Modulight ML7710 Laser is used to deliver a treatment laser beam (modulight.com/ml7710).

In some embodiments, a method comprising: (a) advancing into the bladder a cystoscope comprising a working channel into which an open-ended flexitip ureteral catheter has been inserted; (b) positioning the cystoscope over the tumor midpoint at a distance of about 4 cm from the bladder wall; (c) removing the catheter without moving the cystoscope; (d) inserting a light delivery catheter comprising a 1 cm tip into the working channel of the cystoscope such that the 1 cm tip is positioned outside the cystoscope about 3 cm away from the bladder wall to produce a 2 cm laser spot size, wherein the light delivery catheter comprises an aiming laser and a treatment laser; (e) targeting light at the target tumor using the aiming laser to set the target area; and (f) activating the treatment laser within the target area to deliver a total light dose of about 100 J/cm². In some embodiments the light delivery catheter is a Medlight FD1 Frontal Light Distributor (medlight.com/products/frontal-light-distributor).

In some embodiments, a treatment laser is configured to deliver light (beam) at an irradiance of about 600 mW/cm².

In some embodiments, light from the treatment laser is delivered to the target tumor for a duration of about 140 to about 180 seconds, optionally about 166 seconds.

### BRIEF DESCRIPTION OF THE DRAWINGS

FIG. 1 depicts an exemplary noninvasive injection procedure for delivering a pharmaceutical composition to a target urothelial tumor. Arrows represent trajectory and location of injection.

FIG. 2 depicts an image of a needle with notations, used to deliver a pharmaceutical composition to a target urothelial tumor.

FIG. 3 depicts efficacy data for Ta high-risk NMIBC patients in Cohorts A-C(single-dose drug with light activation). ^{a}For purposes of this analysis, Clinical complete response defined as absence of tumor cells on histopathologic evaluation. ^{b}Bladder urothelial field effect: absence of tumor cells in non-target lesions. ^{c}Previously treated tumor demonstrated high-grade disease but pathology at time of treatment revealed low-grade disease in non-target tumor. ^{d}Local pathology with no evidence of carcinoma in 3/3 target specimens. Central pathology demonstrated single fibrovascular core in 1/3 target specimens consistent with small area of papillary disease of unclear distance from target injection. ^{e}Immune response is defined by immunocyte infiltration on post-treatment histopathology. ^{f}Single lesion visualized at screening on office cystoscopy. Multiple lesions subsequently seen with improved visualization at time of TURBT qualifying for intermediate risk classification.

FIG. 4 depicts efficacy data for Ta intermediate-risk NMIBC patients in Cohorts A-C (single-dose drug with light activation). ^{a}Clinical complete response defined as absence of tumor cells on histopathologic evaluation. ^{b}Bladder urothelial field effect: absence of tumor cells in non-target lesions. ^{c}Immune response is defined by immunocyte infiltration on post-treatment histopathology. ^{d}Two tumors in target tumor field with 1/2 tumors with clinical complete response. BCG failure qualifying as high risk by American Urological Association (AUA) or International Bladder Cancer Group (IBCG) criteria.

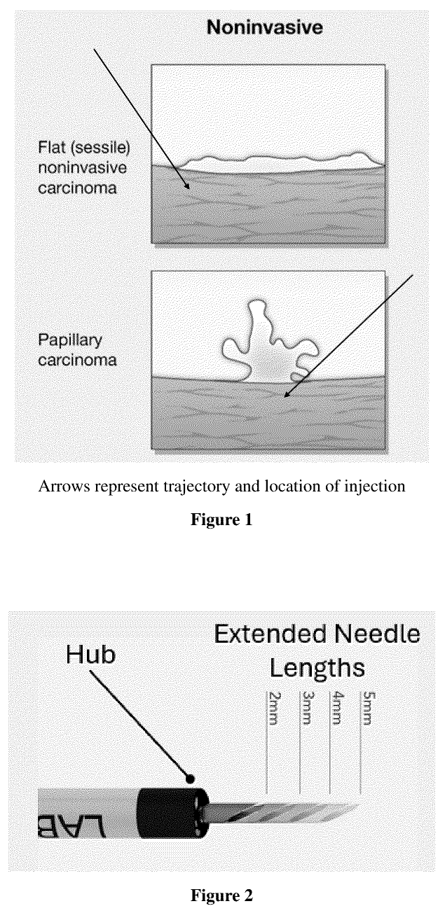

### DETAILED DESCRIPTION

Provided herein, in some aspects, is a front-line therapy for cancer, for example, urothelial cancer, including bladder cancer, such as non-muscle invasive bladder cancer (NMIBC). Without being bound by therapy, the treatments of the disclosure, based on virus-related particles, have a dual mechanism of action and can reduce the treatment burden by disrupting the tumor cell membrane and inducing pro-immunogenic cell death (e.g., via necrosis), leading to T cell activation and immune-mediated tumor cell killing.

The treatments provided herein address the high unmet medical need for function-preserving organ-sparing therapies. Bladder cancer, as a non-limiting example, is the 9^{th} most common cancer worldwide, with more than 600,000 cases per year globally (ranked the 13^{th} for mortality). Conventional bladder cancer treatments are suboptimal, often leading to short- and long-term local and distant side effects, considerable impact on quality of life, inadequate efficacy, multiple surgeries, disease progression, and ultimately loss of the bladder in patients progressing to muscle-invasive bladder cancer (MIBC). The goals of the treatments described herein are to, for example, provide a focal treatment with direct tumor cell killing, provide a front-line early intervention for local disease, stimulate a broad anti-tumor T cell response, decrease treatment burden with a highly favorable safety profile, reduce risk of recurrence and progression, and avoid surgeries, such as transurethral resection of bladder tumor (TURBT) and cystectomy.

Some aspects of relate to methods that can be performed as an in-office procedures in under 15 minutes of total procedure time, requiring no more than local cystoscopic administration of the pharmaceutical composition (e.g., using a needle similar to/the same as that used for BOTOX® injections), and in some embodiments in which light activation is required, laser application.

### Target Lesions

Methods provided herein include delivering to the base of a target tumor, or to a site proximate to the base of the target tumor, a pharmaceutical composition comprising a therapeutic agent in a therapeutically effective amount sufficient to treat the target tumor and induce a therapeutic field effect. A target lesion includes any abnormal growth or change in the target organ or tissue, such as the epithelial lining of the of the target organ or tissue. Target lesions can vary widely in nature, and they can be benign, premalignant, or malignant (cancerous). In some embodiments, a target lesion is a benign lesion, such as a papilloma or cystitis. In some embodiments, a target lesion is a premalignant benign lesion. In some embodiments, a target lesion is a premalignant lesion. In some embodiments, a target lesion is a malignant lesion.

In some aspects, methods provided herein include delivering to the base of a target urothelial tumor, or to a site proximate to the base of the target urothelial tumor, a pharmaceutical composition (e.g., comprising a virus-related particle) in a therapeutically effective amount sufficient to treat the target urothelial tumor and induce a urothelial field effect (e.g., a bladder urothelial field effect). A urothelial lesion includes any abnormal growth or change in the urothelium, which is the epithelial lining of the urinary tract, including the bladder, ureters, and parts of the kidneys. Urothelial lesions can vary widely in nature, and they can be benign, premalignant, or malignant (cancerous). In some embodiments, a urothelial lesion is a benign urothelial lesion, such as a papilloma or cystitis. In some embodiments, a urothelial lesion is a premalignant benign lesion, such as a papillary neoplasm of low malignant potential (PNLMP). In some embodiments, a urothelial lesion is a premalignant lesion, such as urothelial carcinoma in situ (CIS). In some embodiments, a urothelial lesion is a malignant urothelial lesion, such as urothelial carcinoma.

A “target” tumor (or lesion) refers to a specific tumor (or lesion) identified as the primary focus of treatment, and herein, specifically refers to the tumor to which a pharmaceutical composition is directly delivered (e.g., injected at the base of or proximate to the base of the tumor). In some embodiments, the target tumor is in an organ or tissue. Non-limiting examples of organs or tissues are skin, colorectal, breast, ovarian, prostate, lung, esophageal, hepatocellular, liver, pancreatic, head and neck, bladder, and urothelial. In some embodiments, the target tumor is a urothelial tumor (or lesion). Contrast this to a “non-target” tumor (or lesion), which is an additional tumor in the same organ or tissue to which the pharmaceutical composition is not directly delivered (e.g., injected). In some embodiments, the non-target tumor is a non-target urothelial tumor, which is an additional tumor in the urothelium to which the pharmaceutical composition is not directly delivered (e.g., injected). As the data herein unexpectedly demonstrates, delivery of a pharmaceutical composition comprising a therapeutic agent (e.g., a virus-related particle) directly to the base of or proximate to the base of a target (e.g., urothelial) tumor not only treats the target tumor but also, in some embodiments, treats one or more non-target (e.g., urothelial) tumors located away from (e.g., at least 2 cm away from) the target (e.g., urothelial) tumor.

In some embodiments, a urothelial lesion (e.g., urothelial tumor) is in the urinary bladder. In some embodiments, a urothelial lesion is a bladder cancer (e.g., bladder cancer lesion). Bladder cancer is categorized by the location and the level of invasion of a tumor in the bladder wall. The bladder wall comprises four layers from the inside (the lumen) to the outside of the bladder: the mucosa, submucosa, muscularis, and serosa/adventitia. The innermost mucosa is made up of transitional epithelium, allowing the bladder to stretch as it fills with urine. The mucosa layer includes the urothelium (part of the innermost mucosa layer, specifically the transitional epithelial lining) and the lamina propria (lies just beneath the urothelium, serving as a supportive connective tissue). Beneath the mucosa layer lies the submucosa, a layer of connective tissue providing structural support. The muscularis (also referred to as detrusor muscle), is a thick layer of smooth muscle responsible for bladder contractions during urination. Finally, the outermost layer is either serosa (a thin membrane covering the superior surface) or adventitia (fibrous connective tissue) on the other surfaces.

Bladder cancer is divided into two major groups: NMIBC and muscle invasive bladder cancer (MIBC). Examples of further categorizations of NMIBC include, but are not limited to, carcinoma in situ (bladder cancer stage “Tis”), non-invasive papillary carcinoma (bladder cancer stage “Ta”), and carcinomas that have invaded the lamina propria connective tissue (bladder cancer stage “T1”). Examples of further categories of MIBC include, but are not limited to, carcinomas that have invaded the superficial muscle tissue (bladder cancer stage “T2a”), carcinomas that have invaded the deep muscle tissue (bladder cancer stage “T2b”), carcinomas that have invaded the perivesical tissue (bladder cancer stage “T3”), and carcinomas that have invaded adjacent tissue and organs (bladder cancer stage “T4”).

In some embodiments, the present disclosure provides methods for treatment of urothelial cancer of the bladder (UCB), also known as transitional cell carcinoma, which is the most common type of bladder cancer, originating in the urothelial cells that line the inside of the bladder. These urothelial cells are unique because they can stretch as the bladder fills and contracts when it empties. UCB typically begins in the innermost layer of the bladder wall, the urothelium, and can progress through the other layers if not detected early. It can range from non-invasive (confined to the urothelium) to invasive (penetrating deeper into the bladder's muscular or connective layers), making early detection crucial for better outcomes.

In some embodiments, the present disclosure provides methods for treatment of the NMIBC subtypes. In some embodiments, the NMIBC is a carcinoma in situ. In some embodiments, the NMIBC is a non-invasive papillary carcinoma. In some embodiments, the NMIBC is a tumor that has invaded the lamina propria connective tissue. NMIBC tumors to be treated in accordance with the present disclosure are typically about 3 centimeters in diameter but may be smaller or larger. In some embodiments, a subject being treated for bladder cancer has a tumor with a diameter of about 0.5 cm to about 5 cm. In some embodiments, the tumor is about 1 cm to about 4 cm. In some embodiments, the tumor is about 0.5 cm, 1 cm, 1.5 cm, 2 cm, 2.5 cm, 3 cm, 3.5 cm, 4 cm, 4.5 cm, or 5 cm.

Bladder cancers may also be classified as bacillus Calmette-Guerin (BCG) experienced/exposed or BCG refractory/unresponsive bladder cancer. The term “BCG experienced/exposed” simply indicates that the patient has undergone BCG therapy, meaning they have been exposed to BCG treatment for non-muscle-invasive bladder cancer (NMIBC), for example. Being BCG experienced doesn't imply anything about the patient's response to the treatment—it just means they have received it. Some patients may have a positive response, where BCG helps control or eradicate the cancer. The term “BCG refractory/unresponsive” is used for patients who have undergone BCG therapy, but the treatment has failed to control the disease. BCG refractory refers to patients whose bladder cancer has persisted or recurred within 6 months of receiving BCG therapy, indicating that the cancer is not responding to the treatment.

BCG unresponsive is a broader term that includes patients who either do not respond to BCG initially (refractory) or have a relapse after achieving an initial complete response but relapse later during treatment. Essentially, BCG refractory/unresponsive patients are those for whom BCG is no longer an effective option, whereas BCG experienced/exposed patients may still be benefiting from or have completed BCG treatment with varying outcomes. In some embodiments, a subject refractory/unresponsive is a BCG experienced/exposed patient. In some embodiments, a subject refractory/unresponsive is a BCG refractory/unresponsive patient.

In some embodiments, a human subject is a patient diagnosed with a low-risk urothelial cancer (e.g., single, low-grade tumor, a “low risk patient”). In some embodiments, a human subject is a patient diagnosed with an intermediate-risk urothelial cancer (e.g., recurrent or bigger low-grade tumors or—depending on the risk classification applied-Ta high-grade tumors, an “intermediate risk patient”). In some embodiments, an intermediate risk patient is diagnosed with intermediate NMIBC. In some embodiments, an intermediate risk patient is diagnosed with intermediate MIBC. In other embodiments, a human subject is a patient diagnosed with high-risk urothelial cancer (e.g., high-grade, a “high risk patient”). In some embodiments, a high risk patient is diagnosed with high risk NMIBC. In some embodiments, a high risk patient is diagnosed with high risk MIBC. In yet other embodiments, a human subject is a patient diagnosed with high-risk carcinoma in situ (CIS) and/or is unresponsive to BCG therapy.

### Pharmaceutical Compositions

Pharmaceutical compositions of the disclosure include a therapeutic agent. A “therapeutic agent” includes any agent that can be useful for treating a target tumor. In some embodiments, a therapeutic agent is an anticancer (e.g., antitumor) agent. An anticancer agent includes an agent that inhibits the growth of a tumor or otherwise causes cell death of tumor cells. In other embodiments, the therapeutic agent is an immune modulating (e.g., immunomodulatory) agent. An immune modulating agent suppresses or activates an immune response. In some embodiments, an immune modulating agent (e.g., immunostimulatory agent) is used in combination with an anticancer agent.

Therapeutic agents, for example, anticancer agents and/or immunomodulating agents, come in many forms, for example, polypeptides, polynucleotides, and small molecules. In some embodiments, a therapeutic agent is an antibody (e.g., a monoclonal antibody). In some embodiments, a therapeutic agent is an antigen-binding agent or antigen-binding fragment. In some embodiments, a therapeutic agent is a drug conjugate (e.g., a drug covalently or non-covalently linked to a targeting molecule), such as an antibody drug conjugate, peptide drug conjugate, or viral drug conjugate (e.g., virus-like drug conjugate). In some embodiments, the therapeutic agent is a vaccine. In some embodiments, the therapeutic agent is a gene therapy vector, e.g., adeno-associated virus (AAV) or lentiviral vector encoding an anticancer or immunomodulatory gene of interest. In some embodiments, the therapeutic agent is an oncolytic virus. In some embodiments, the therapeutic agent is an immunostimulatory agent.

In some embodiments, an immunomodulating agent or immunostimulatory agent is a checkpoint inhibitor. Immune checkpoints are proteins in the immune system that either enhance an immune response signal (co-stimulatory molecules) or reduce an immune response signal. Many cancers protect themselves from the immune system by exploiting the inhibitory immune checkpoint proteins to inhibit the T cell signal. Such inhibitory checkpoint proteins include, without limitation, Cytotoxic T-Lymphocyte-Associated protein 4 (CTLA-4), Programmed Death 1 receptor (PD-1), Programmed Death-Ligand 1 (PD-L1), T-cell Immunoglobulin domain and Mucin domain 3 (TIM3), Lymphocyte Activation Gene-3 (LAG3), V-set domain-containing T-cell activation inhibitor 1 (VTVN1 or B7-H4), Cluster of Differentiation 276 (CD276 or B7-H3), B and T Lymphocyte Attenuator (BTLA), Galectin-9 (GAL9), Checkpoint kinase 1 (Chk1), Adenosine A2A receptor (A2aR), Indoleamine 2,3-dioxygenase (IDO), Killer-cell Immunoglobulin-like Receptor (KIR), Lymphocyte Activation Gene-3 (LAG3) and V-domain Ig suppressor of T cell activation (VISTA).

In some embodiments, the therapeutic agent is an antibody drug conjugate. Non-limiting examples of antibody drug conjugates used to treat bladder cancer include enfortumab vedotin-ejfv and sacituzumab govitecan. Other examples of antibody drug conjugates include cetuximab-IR700, panitumumab-IR700, trastuzumab-IR700, BMS-935559-IR700, MEDI4736-IR700, MPDL3280A-IR700, MSB0010718C-IR700, and ASP-1929. Non-limiting examples of drug conjugates for use with the methods provided herein are described in International Publication Nos. WO2015042325A1, WO2015187677A1, WO2015187651A1, WO2017031367A1, and WO2018156815A1.

Other conjugates are contemplated herein. For example, a conjugate may include a targeting molecule (a molecule that binds to a protein or other moiety on the surface of a cell) selected from a NANOBODY® molecule, an AFFIBODY® molecule, a diabody, a minibody, an antigen, a ligand, a protein, a peptide, a nucleic acid and a small molecule conjugated to a therapeutic agent, such as a photosensitizer.

In some embodiments, a conjugate comprises a first therapeutic agent (e.g., photosensitizer), a targeting molecule (e.g., antibody), and a second therapeutic agent (e.g., anticancer agent).

The components of the conjugates described herein may be covalently linked or non-covalently linked to each other. In some embodiments, the conjugates comprise a linker, for example, a peptide linker, a polypeptide linker, or a chemical linker. The linker may be cleavable, in some embodiments, for example cleavable in a tumor microenvironment (e.g., by a matrix metalloproteinase (MMP) present in the tumor microenvironment).

In some embodiments, the therapeutic agent is a virus-related particle. A “virus-related particle” includes any entity that comprises a viral protein. The term encompasses viruses as well as non-infectious particles that are structurally similar to a virus but may not have all the components or properties of a fully functional virus (e.g., lack the ability to replicate independently). Non-limiting examples of virus-related particles include viruses, virus-like particles, defective viral particles, and pseudoviruses.

In some embodiments, a virus-related particle is a virus. Non-limiting examples of viruses that may be used as provided herein include adenoviruses, adeno-associated viruses, herpes simplex viruses, lentiviruses, and retroviruses. In some embodiments, a virus is an oncolytic viruses (e.g., measles virus, reovirus, vaccinia virus, talimogene laherparepvec, adenovirus, etc.).

In some embodiments, a virus-related particle is a replication incompetent virus. Replication-incompetent viruses include viruses that have been genetically modified so they cannot replicate on their own within host cells. In some embodiments, a replication incompetent virus is selected from adenoviruses, adeno-associated viruses, herpes simplex viruses, lentiviruses, and retroviruses. In some embodiments, a replication incompetent virus is an oncolytic viruses.

In some embodiments, a virus-related particle is a virus-like particle (VLP). A virus-like particle includes a non-infectious particle that mimics the structure of a virus by including the other protein capsid of a virus but lacks viral genetic material. A “papillomavirus (PV) VLP” is a VLP that includes L1 and/or L2 capsid proteins from a PV. In some embodiments, a PV VLP includes PV L1 and L2 capsid proteins that “self-assemble” into a 55 nM VLP without incorporating the viral genome. Such PV VLPs can bind cancer cells with high selectivity both in vitro and in vivo through a mechanism of action (MoA) that involves the initial binding to a subset of modified tumor-associated glycosaminoglycans (GAGs) on the tumor cell membrane as well as the basement membrane. PVs have a unique tropism towards cancer cells that is mediated by the binding of the viral capsid to specific modifications of GAGs on the surface of the cancer cells that are not normally found on healthy benign cells. In some embodiments, a VLP is a human papillomavirus (HPV) VLP. An HPV VLP can have, for example, HPV L1 capsid proteins, HPV L2 capsid proteins, or a combination of HPV L1 capsid proteins and HPV L2 capsid proteins.

In some embodiments, the HPV L1 capsid proteins comprises the sequence of SEQ ID NO: 1:
MSLWLPSEATVYLPPVPVSKVVSTDEYVARTNIYYHAGTSRLLAVGHPYFPIKKPNN NKILVPKVSGLQYRVFRIHLPDPNKFGFPDTSFYNPDTQRLVWACVGVEVGRGQPLGVGIS GHPLLNKLDDTENASAYAANAGVDNRECISMDYKQTQLCLIGCKPPIGEHWGKGSPCTNV AVNPGDCPPLELINTVIQDGDMVDTGFGAMDFTTLQANKSEVPLDICTSICKYPDYIKMVS EPYGDSLFFYLRREQMFVRHLFNRAGAVGENVPTDLYIKGSGSTATLANSNYFPTPSGSMV TSDAQIFNKPYWLQRAQGHNNGICWGNQLFVTVVDTTRSTNMSLCAAISTSETTYKNTNF KEYLRHGEEYDLQFIFQLCKITLTADVMTYIHSMNSTILEDWNFGLQPPPGGTLEDTYRFVT SQAIACQKHTPPAPKEDPLKKYTFWEVNLKEKFSADLDQFPLGRKFLLQAGLKAKPKFTLG KRKATPTTSSTSTTAKRKKRKL (SEQ ID NO: 1), or a sequence having at least 80%, at least 85%, at least 90%, or at least 95% identity thereto.

In some embodiments, the HPV L2 capsid proteins comprise the sequence of SEQ ID NO: 2:
MRHKRSAKRTKRASATQLYKTCKQAGTCPPDIIPKVEGKTIADQILQYGSMGVFFGGLGIG TGSGTGGRTGYIPLGTRPPTATDTLAPVRPPLTVDPVGPSDPSIVSLVEETSFIDAGAPTSVPSI PPDVSGFSITTSTDTTPAILDINNTVTTVTTHNNPTFTDPSVLQPPTPAETGGHFTLSSSTISTH NYEEIPMDTFIVSTNPNTVTSSTPIPGSRPVARLGLYSRTTQQVKVVDPAFVTTPTKLITYDNP AYEGIDVDNTLYFSSNDNSINIAPDPDFLDIVALHRPALTSRRTGIRYSRIGNKQTLRTRSGKSI GAKVHYYYDLSTIDPAEEIELQTITPSTYTTTSHAASPTSINNGLYDIYADDFITDTSTTPVPS VPSTSLSGYIPANTTIPFGGAYNIPLVSGPDIPINITDQAPSLIPIVPGSPQYTIIADAGDFYLHPS YYMLRKRRKRLPYFFSDVSLAA (SEQ ID NO: 2), or a sequence having at least 80%, at least 85%, at least 90%, or at least 95% identity thereto.

In some embodiments, a virus-related particle is a virus-like drug conjugate. A virus-like drug conjugate can include, for example, a VLP conjugated to a therapeutic agent (e.g., a cytotoxic agent). Non-limiting examples of therapeutic agents that may be used include chemotherapeutic agents (e.g., doxorubicin, paclitaxel, gemcitabine), small molecule inhibitors (e.g., tyrosine kinase inhibitors, proteosome inhibitors), nucleic acids (e.g., siRNA, miRNA, antisense oligonucleotides), radioactive isotopes, enzymes, and dyes.

In some embodiments a virus-like drug conjugate comprises a photosensitizer. A photosensitizer includes a chemical compound that becomes activated when exposed to a specific wavelength of light, resulting in a chemical reaction that can produce reactive oxygen species (ROS) or other reactive molecules. These ROS can cause damage to nearby cells or tissues. Non-limiting examples of photosensitizers include porphyrins, aminolevulinic acid, and phthalocyanines. Additional examples of photosensitizers include HpD, porfimer sodium (PHOTOFRIN®, PHOTOGEM®, PHOTOSAN HEMPORFIN®), m-THPC, Temoporfin (Foscan®), Verteporfin (VISUDYNE®), HPPH (PHOTOCHLOR®), Palladium-bacteria-pheophorbide (TOOKAD®,) 5-ALA, 5 aminolevulinic acid (LEVULAN®), 5-ALA methylester (METVIX®), 5-ALA benzylester (BENZVIX®), 5-ALA hexylester (HEXVIX®), lutetium (III)-texaphyrin or Motexafin-lutetium (LUTEX®, LUTRIN®, ANGRIN®, OPTRIN®), SnET2, Tin (IV) ethyl etiopurpurin (PURLYTIN®, PHOTREX®), NPe6, mono-L-aspartyl chlorine e6, talaporfin sodium (TALPORFIN®, LASERPHYRIN®), BOPP, boronated protoporphyrin (BOPP®), Zinc phthalocyanine (CGP55847®), silicon phthalocyanine (Pc4®), mixture of sulfonated aluminium phthalocyanine derivatives (PHOTOSENS®), ATMPn, Acetoxy-tetrakis (beta-methoxyethyl-)porphycene), TH9402 and dibromorhodamine methyl ester. Examples of photosensitizing dyes for use in accordance with the present disclosure include those that can be used in fluorescence imaging (e.g., near infrared (NIR) fluorescent dyes) such as LA JOLLA BLUE® and IRDye® 700DX.

In some embodiments, a virus-like drug conjugate comprises a phthalocyanine. Non-limiting examples of phthalocyanines include aluminum phthalocyanine chloride, silicon phthalocyanine, zinc phthalocyanine, copper phthalocyanine, and infrared or near-infrared dyes. In some embodiments, a virus-like drug conjugate comprises a phthalocyanine dye component (e.g., an infrared or near-infrared dye) that catalyzes the production of reactive oxygen species. This results in a potent and selective acute tumor cell necrosis without harming surrounding healthy cells.

In some embodiments a virus-like drug conjugate comprises an infrared dye of Formula I (e.g., IRDye® 700DX carboxylate):

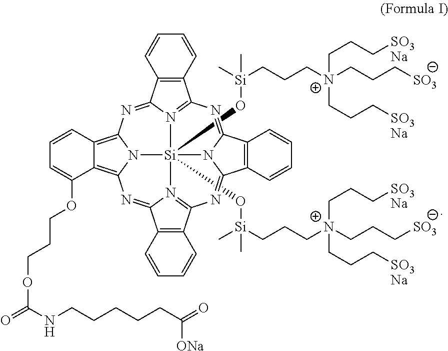

In some embodiments a virus-like drug conjugate comprises an infrared dye of Formula II (e.g., IRDye® 700DX N-hydroxysuccinimide (NHS) ester):

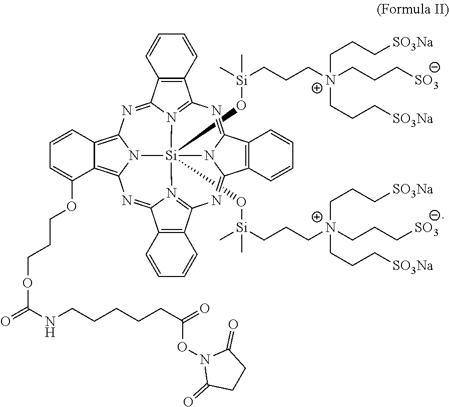

In some embodiments, a virus-like drug conjugates comprises about 100 to about 1000 molecules of a therapeutic agent, for example, an infrared dye of Formula I or Formula II (“dye molecules”). For example, a virus-like drug conjugate can comprise about 100 to about 500, about 100 to about 400, about 100 to about 300 molecules of a therapeutic agent. In some embodiments, a virus-like drug conjugate comprises about 200 molecules of a therapeutic agent, e.g., an infrared dye of Formula I or Formula II.

In some embodiments, a virus-like drug conjugate comprises a human papillomavirus (HPV) virus-like particle comprising HPV L1 and L2 capsid proteins (e.g., respectively comprising the amino acid sequence of SEQ ID NO: 1 and SEQ ID NO: 2), wherein the capsid proteins are conjugated to about 100 to about 300 (e.g., about 200) molecules of Formula I or Formula 2. In some embodiments, a pharmaceutical composition comprises: (a) a virus-like drug conjugate comprises a human papillomavirus (HPV) virus-like particle comprising HPV L1 and L2 capsid proteins (e.g., respectively comprising the amino acid sequence of SEQ ID NO: 1 and SEQ ID NO: 2), wherein the capsid proteins are conjugated to about 100 to about 300 (e.g., about 200) molecules of Formula I or Formula 2; and (b) an isotonic buffer, for example, having a pH of about 6.5.

In some embodiments, a virus-like drug conjugate has the structure of:

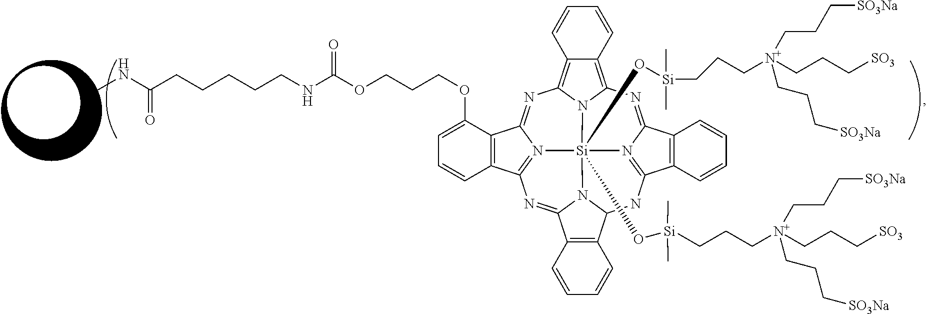

wherein the gray circle to the left represents an empty proteinaceous sphere of approximately 55 nm in diameter, composed of 72 capsomeres that are made of ˜5 HPV capsid protein L1 (e.g., SEQ ID NO: 1) and up to ˜1 HPV capsid protein L2 (e.g., SEQ ID NO: 2).

In some embodiments, a pharmaceutical composition is referred to as “bel-sar,” used interchangeably with “belzupacap sarotalocan.” Bel-sar is an isotonic solution comprising a recombinantly manufactured virus-like drug conjugate comprising (a) an empty proteinaceous virus-like particle having a diameter of about 55 nm and including about 72 capsomeres, wherein each capsomere includes about five modified HPV L1 capsid proteins (SEQ ID NO: 1) and about one HPV L2 capsid protein (SEQ ID NO: 2); and (b) about 200 molecules of Formula I (IRDye® 700DX) conjugated to the capsid proteins of the virus-like particle. A VLP resembles an empty viral capsid, which includes a protein shell made of capsomeres. “Capsomere” refers to a subunit of a viral capsid (or VLP) made of capsid proteins.

A “therapeutically effective amount” of a pharmaceutical composition herein general refers to the quantity of the viral-related particle (e.g., virus-like drug conjugate) sufficient to achieve a desired therapeutic effect, such as inhibiting tumor growth, reducing tumor size, eliminating cancer cells, inducing a therapeutic field effect, and/or transforming a cold tumor into a hot tumor.

The pharmaceutical compositions of the disclosure, in some embodiments, are administered in a therapeutically effective amount sufficient to treat a target (e.g., urothelial) tumor. “Treat,” “treating,” or “treatment.” in the context of a tumor (target or non-target, e.g., target urothelial or non-target urothelial) herein includes immune activation, inhibiting tumor growth, reducing tumor size (tumor shrinkage), and/or histopathological evidence of cancer cell absence (partial or complete) or necrosis.

The pharmaceutical compositions of the disclosure, in some embodiments, are administered in a therapeutically effective amount sufficient to induce a therapeutic field effect. Induction of a “therapeutic field effect” refers to the induction (e.g., initiation) of an immune response in a region that includes a (one or more) non-target tumor (e.g., separate and some distance away from the target tumor) and/or a reduction in the number of cancerous cells in a non-target tumor. In some embodiments, the reduction in the number of cancer cells is the result of cell death (e.g., necrosis). In some embodiment, delivery of a pharmaceutical composition of the disclosure to a target tumor results in treatment (e.g., reduction in tumor volume and/or cancer cell death) of the target tumor and treatment (e.g., reduction in tumor volume and/or cancer cell death) of one or more non-target tumors.

In some embodiments, the therapeutic field effect is a “urothelial field effect” or a bladder urothelial field effect” wherein the induction (e.g., initiation) of an immune response in a region that includes a (one or more) non-target urothelial tumor (e.g., separate and some distance away from the target urothelial tumor) and/or a reduction in the number of cancerous cells in a non-target urothelial tumor. In some embodiments, the reduction in the number of cancer cells is the result of cell death (e.g., necrosis). In some embodiment, delivery of a pharmaceutical composition of the disclosure to a target urothelial tumor results in treatment (e.g., reduction in tumor volume and/or cancer cell death) of the target urothelial tumor and treatment (e.g., reduction in tumor volume and/or cancer cell death) of one or more non-target urothelial tumors (e.g., in the bladder). Surprisingly, the clinical data provided herein demonstrates that this therapeutic field effect (e.g., bladder urothelial field effect) is induced in human patients in the absence of systemic exposure of the virus-related particle (e.g., bel-sar) and in the absence of local or systemic exposure of an immune checkpoint inhibitor (e.g., anti-PD-1, and i-PD-L1, anti-CTLA4, or anti-LAG-3 antibodies). As is understood in the field, “system exposure” refers to the distribution of a substance, such as a virus-related particle herein, throughout the body after it enters the bloodstream.

The pharmaceutical compositions of the disclosure, in some embodiments, are administered in a therapeutically effective amount sufficient to transform a cold tumor into a hot tumor. A cold tumor includes a type of tumor that is immunologically inactive, meaning it contains few or no infiltrating immune cells, particularly cytotoxic T lymphocytes (CTLs). These tumors often have a suppressive tumor microenvironment characterized by poor antigen presentation, low levels of inflammatory cytokines, and the presence of immunosuppressive cells such as regulatory T cells (Tregs) and myeloid-derived suppressor cells (MDSCs). As a result, cold tumors typically do not respond well to immunotherapies, such as immune checkpoint inhibitors. In contrast, a hot tumor includes an immunologically active tumor that shows robust infiltration of immune cells, especially CD8 T cells, and exhibits signs of immune activation and inflammation within the tumor microenvironment. Hot tumors often express tumor antigens and interferon-related gene signatures, leading to stronger immune recognition. Because of their active immune milieu, hot tumors are generally more responsive to immunotherapies, particularly checkpoint inhibitors like anti-PD-1. anti-PD-L1, anti-CTLA-4, and/or anti-LAG3 antibodies.

The pharmaceutical compositions of the disclosure, in some embodiments, further comprise a light-activated moiety. A “light-activated moiety” includes any molecule or molecular entity that comprises a functional group that can be activated by a specific wavelength of light. The term encompasses molecules that may become visualized, fluorescent and/or cytotoxic at a specific wavelength of light. In some embodiments, a light-activated moiety is a photosensitizer (e.g., cytotoxic). In some embodiments, a light-activated moiety is used for visualization of the location of the pharmaceutical composition following administration (e.g., delivery) and is not a photosensitizer (e.g. not cytotoxic).

In some embodiments, a light-activated moiety may become visualized and/or fluorescent at one specific wavelength of light and may become cytotoxic at another specific wavelength of light, wherein the specific wavelengths of light are different.

In some embodiments, a pharmaceutical composition of the disclosure comprises one light activated moiety. In some embodiments, the one light activated moiety becomes visualized and/or fluorescent at a specific wavelength of light. In some embodiments, the one light activated moiety becomes cytotoxic at a specific wavelength of light. In some embodiments, the one light activated moiety becomes visualized and/or fluorescent and cytotoxic at a specific wavelength of light.

In some embodiments, a pharmaceutical composition of the disclosure could comprise two light activated moieties, wherein one light activated moiety becomes visualized and/or fluorescent at a specific wavelength of light and another light activated moiety becomes cytotoxic at the same or different specific wavelength of light. In some embodiments, two light activated moieties are activated at the same specific wavelength. In some embodiments, two light activated moieties are activated at different specific wavelengths.

In some embodiments, a therapeutic agent of the disclosure comprises one light activated moiety. In some embodiments, the one light activated moiety becomes visualized and/or fluorescent at a specific wavelength of light. In some embodiments, the one light activated moiety becomes cytotoxic at a specific wavelength of light. In some embodiments, the one light activated moiety becomes visualized and/or fluorescent and cytotoxic at a specific wavelength of light.

In some embodiments, a therapeutic agent of the disclosure could comprise two light activated moieties, wherein one light activated moiety becomes visualized and/or fluorescent at a specific wavelength of light and another light activated moiety becomes cytotoxic at the same or different specific wavelength of light. In some embodiments, two light activated moieties (e.g., one visualized/fluorescent and one cytotoxic) are activated at the same specific wavelength. In some embodiments, two light activated moieties (e.g., one visualized/fluorescent and one cytotoxic) are activated at different specific wavelengths.

### Methods of Administration and Methods of Bladder Cancer Therapy

The methods provided herein include, in some aspects, delivering to the base of a target urothelial tumor a pharmaceutical composition comprising a virus-related particle in a therapeutically effective amount sufficient to treat the target urothelial tumor and induce a bladder urothelial field effect. In other aspects, the methods provided herein include delivering to the base of a target urothelial tumor a pharmaceutical composition comprising a virus-related particle in a therapeutically effective amount sufficient to transform a cold (immunologically inactive) tumor into a hot (immunologically active) tumor. The typical mode of administration (also used interchangeable with the term “delivery”) is injection, but other modes of administration are contemplated herein, provided the mode of delivery results in deposition of the virus-related particle into the base of the target urothelial tumor.

In some embodiments, a needle is used to inject a pharmaceutical composition. Examples of needles include, but are not limited to, hollow needles, coated needles, minineedles or microneedles, depending on the volume of injection fluid and/or size of the injection site. A non-limiting example of a needle that may be used in accordance with the disclosure is depicted in FIG. 2. In some embodiments, the tip of a needle (the needle tip extension) has a length of about 2-4 mm. In some embodiments, the tip of a needle (the needle tip extension) has a length of about 2-5 mm. In some embodiments, the tip of a needle (the needle tip extension) has a length of 2 mm. In some embodiments, the tip of a needle (the needle tip extension) has a length of 3 mm. In some embodiments, the tip of a needle (the needle tip extension) has a length of 4 mm. In some embodiments, the tip of a needle (the needle tip extension) has a length of 5 mm.

In some embodiments, the needle is a Laborie injeTAK® needle. The Laborie injeTAK® needle is a cystoscopy injection needle engineered for precision and safety in intradetrusor drug delivery, for example. It features an adjustable tip that allows one to set the needle depth at 0, 2, 3, 4, or 5 mm, for example, to match the bladder wall thickness, enhancing accuracy and reducing the risk of over-penetration. The retractable needle design protects both the scope and tissue during insertion and removal, while a black indicator tip ensures visual confirmation of placement. Designed for single-handed use, the needle is compatible with both rigid and flexible cystoscopes.

A cystoscope may be used, in some embodiments, to guide delivery of the pharmaceutical composition to the base of the target urothelial tumor. A cystoscope, which includes a channel that houses small instruments, such as a light delivery catheter, a light, camera and/or needle, is usually inserted into the urethra and passed into the bladder lumen. Use of a cystoscope permits visualization, light or laser administration, and/or injection capabilities during the medical procedures that employ the methods described herein.

In accordance with the present disclosure, a pharmaceutical composition may be administered to the base of a target urothelial tumor. The “base” of a target urothelial tumor includes the part of the tumor that is in contact with the surrounding tissue or the underlying structure where the tumor originates. In some embodiments, a pharmaceutical composition is delivered to a site proximate to the base of the target urothelial tumor. A site is considered “proximate to” the base of a target urothelial tumor if the site is within 2 cm of the base of the target urothelial tumor (e.g., within 2 cm of the border of the base of the target tumor). In some embodiments, a pharmaceutical composition is delivered within 1.5 cm of the base of the target urothelial tumor (e.g., within 1.5 cm of the border of the base of the target urothelial tumor). In some embodiments, a pharmaceutical composition is delivered within 1 cm of the base of the target urothelial tumor. In some embodiments, a pharmaceutical composition is delivered within 0.5 cm of the base of the target urothelial tumor (e.g., within 0.5 cm of the border of the base of the target urothelial tumor). In some embodiments, a pharmaceutical composition is delivered within 5 mm of the base of the target urothelial tumor (e.g., within 5 mm of the border of the base of the target urothelial tumor). In some embodiments, a pharmaceutical composition is delivered within 4 mm of the base of the target urothelial tumor (e.g., within 4 mm of the border of the base of the target urothelial tumor). In some embodiments, a pharmaceutical composition is delivered within 3 mm of the base of the target urothelial tumor (e.g., within 3 mm of the border of the base of the target urothelial tumor). In some embodiments, a pharmaceutical composition is delivered within 2 mm of the base of the target urothelial tumor (e.g., within 2 mm of the border of the base of the target urothelial tumor). In some embodiments, a pharmaceutical composition is delivered within 1 mm of the base of the target urothelial tumor (e.g., within 1 mm of the border of the base of the target urothelial tumor). In some embodiments, a pharmaceutical composition is delivered no more than 2 cm away from the base of the target urothelial tumor (e.g., no more than 2 cm away from the border of the base of the target urothelial tumor). In some embodiments, a pharmaceutical composition is delivered no more than 1.5 cm away from the base of the target urothelial tumor (e.g., no more than 1.5 cm away from the border of the base of the target urothelial tumor). In some embodiments, a pharmaceutical composition is delivered no more than 1 cm away from the base of the target urothelial tumor (e.g., no more than 1 cm away from the border of the base of the target urothelial tumor). In some embodiments, a pharmaceutical composition is delivered no more than 0.5 cm (5 mm) away from the base of the target urothelial tumor (e.g., no more than 0.5 cm away from the border of the base of the target urothelial tumor). In some embodiments, a pharmaceutical composition is delivered at (into) the border of the base of the target urothelial tumor. The “border of a tumor”, also referred to as the “tumor margin” is the boundary between the tumor tissue and the surrounding normal tissue.

In some embodiments, a pharmaceutical composition is delivered below or around the base of a target tumor and above the muscularis propria of the bladder. In some embodiments, a pharmaceutical composition is delivered to the lamina propria below the base of a target tumor and above the muscularis propria of the bladder.

Also contemplated herein are multiple sites of administration of a pharmaceutical composition. Thus, a pharmaceutical composition may be injected, for example, multiple times into the base of the target urothelial tumor. That is, a particular treatment session may include one injection or a set of multiple injections (e.g., followed by exposure to laser treatment when the agent is, for example, a virus-like drug conjugate). In some embodiments, a pharmaceutical composition is injected multiple times at different injection sites in or proximate to the base of the target urothelial tumor. In some embodiments, a pharmaceutical composition is injected at least once. In some embodiments, a pharmaceutical composition is injected at least twice, at the same injection site or at different injection sites. In some embodiments, a pharmaceutical composition is injected at least three times, at the same injection site or at different injection sites. In some embodiments, a pharmaceutical composition is injected 1, 2, 3, 4, or 5 times at the same injection site or at different injection sites. The number of injections and pattern of injection used to administer a pharmaceutical composition may vary based on the size of the target urothelial tumor. In some embodiments, the pattern of injection includes injections sites even spaced apart along the periphery of the base of a pharmaceutical composition. The distance between injection sites may vary and may be determined by a practitioner depending, in part, on the pharmaceutical composition.

The volume of a pharmaceutical composition (e.g., per injection) administered to the base of a target urothelial tumor may vary but, in some embodiments, is about 100 μl to about 2 mL. In some embodiments, the volume of the pharmaceutical composition for a single injection is about 100 μl to about 1.5 mL. In some embodiments, the volume of the pharmaceutical composition for a single injection is about 100 pl to about 1 mL. In some embodiments, the volume of the pharmaceutical composition for a single injection is about 100 μl to about 500 μl. In some embodiments, the volume of the pharmaceutical composition for a single injection is about 500 pl to about 1.5 mL. In some embodiments, the volume of the pharmaceutical composition for a single injection is about 500 μl to about 1 mL. In some embodiments, the volume of the pharmaceutical composition for a single injection is about 1 mL to about 2 mL. In some embodiments, the volume of the pharmaceutical composition for a single injection is about 100 μl, about 150 μl, about 200 μl, about 250 μl, about 300 μl, about 350 μl, about 400 μl, about 450 μl, about 500 μl, about 550 μl, about 600 μl, about 650 μl, about 700 μl, about 750 μl, about 800 μl, about 850 μl, about 900 μl, about 950 μl, about 1000 μl, about 1250 μl, about 1500 μl, about 1750 μl, or about 2000 μl. In some embodiments, the volume of the pharmaceutical composition for a single injection is about 250 μl. In some embodiments, the volume of the pharmaceutical composition for a single injection is about 500 pl. In some embodiments, the volume of the pharmaceutical composition for a single injection is about 1 mL. In some embodiments, the volume of the pharmaceutical composition for a single injection is about 1.5 ml. In some embodiments, the volume of the pharmaceutical composition for a single injection is about 2 mL.

In some embodiments, about 1 ml of the pharmaceutical composition is injected into a target tumor, wherein the therapeutically effective amount delivered is about 200 μg to about 800 ug. Thus, in some embodiments, the concentration of a pharmaceutical composition is about 0.2 mg/mL (e.g., per tumor). In some embodiments, the concentration of a pharmaceutical composition is about 0.3 mg/mL (e.g., per tumor). In some embodiments, the concentration of a pharmaceutical composition is about 0.4 mg/mL (e.g., per tumor). In some embodiments, the concentration of a pharmaceutical composition is about 0.5 mg/mL (e.g., per tumor). In some embodiments, the concentration of a pharmaceutical composition is about 0.6 mg/mL (e.g., per tumor). In some embodiments, the concentration of a pharmaceutical composition is about 0.7 mg/mL (e.g., per tumor). In some embodiments, the concentration of a pharmaceutical composition is about 0.8 mg/mL (e.g., per tumor). In some embodiments, the concentration of a pharmaceutical composition is about 0.9 mg/mL (e.g., per tumor). In some embodiments, the concentration of a pharmaceutical composition is about 1.0 mg/mL (e.g., per tumor).A non-limiting exemplary injection procedure used to deliver a pharmaceutical composition to a target urothelial tumor is as follows:
Adjust needle to achieve a total length of about 2-5 mm extended beyond the instrument hub (see, e.g., FIG. 2). In some embodiments, the length of the needle is 1 mm. In some embodiments, the length of the needle is 2 mm. In some embodiments, the length of the needle is at least 2 mm. In some embodiments, the length of the needle is 3 mm. In some embodiments, the length of the needle is 4 mm. In some embodiments, the length of the needle is 5 mm. In some embodiments, the length of the needle is no more than 5 mm. In some embodiments, the length of the needle is at least 2 mm and not more than 5 mm.
Identify border between normal urothelium and tumor surface (benign urothelial/tumor border).
Insert needle at the identified benign urothelial/tumor border and advance into the tissue up to the level of the instrument hub (e.g., about 2-5 mm). In some embodiments, the needle is advanced into the tissue about 1 mm. In some embodiments, the needle is advanced into the tissue about 2 mm. In some embodiments, the needle is advanced into the tissue about 3 mm. In some embodiments, the needle is advanced into the tissue about 4 mm. In some embodiments, the needle is advanced into the tissue about 5 mm.
Ensure tip remains above the muscularis propria and within the mucosa (e.g., lamina propria).
Slowly inject the pharmaceutical composition (e.g., at 1-2 cc/min). In some embodiments, the rate of injection is 0.5 cc/min. In some embodiments, the rate of injection is 1 cc/min. In some embodiments, the rate of injection is 1.5 cc/min. In some embodiments, the rate of injection is 2 cc/min. In some embodiments, the rate of injection is 2.5 cc/min. In some embodiments, the rate of injection is 3 cc/min. In some embodiments, the rate of injection is 3.5 cc/min. In some embodiments, the rate of injection is 4.5 cc/min. In some embodiments, the rate of injection is 4.5 cc/min. In some embodiments, the rate of injection is 5 cc/min.
Repeat multiple injections as necessary at comparable spacing along the benign urothelial/tumor border to ensure equal distribution of virus-related particle dose along the target tumor base. In some embodiments, multiple injection includes 2 injections. In some embodiments, multiple injection includes 3 injections. In some embodiments, multiple injection includes 4 injections. In some embodiments, multiple injection includes 5 injections. The number of injections can depend on the size of the target tumor.

As described elsewhere herein, in some embodiment, a virus-related particle comprises a photosensitive molecule, such as photosensitizer, that is “activated” (e.g., becomes cytotoxic) at a specific wavelength of light. A photosensitive molecule is considered “light activated moiety”. Thus, in some embodiments, the methods provide herein comprise delivering (e.g., injecting) a pharmaceutical composition to the base of a target urothelial tumor and/or proximate to base of a target urothelial tumor and exposing the target tumor (e.g., delivery site_ to an excitation wavelength of light (thereby activating the photosensitive molecule). In some embodiments, the therapeutic agent is a photosensitive VDC comprising a virus-like particle conjugated to a photosensitizer (e.g., bel-sar).

In some embodiments, activation of the photosensitive molecule renders it cytotoxic or able to produce a cytotoxic molecule such as reactive oxygen species (i.e., photosensitizers). Suitable wavelengths of light include, without limitation, ultraviolet wavelengths, visible wavelengths, infrared wavelengths and near infrared wavelengths. In some embodiments, photosensitizers are activated and become cytotoxic at a wavelength of 600 nm to 800 nm, or 660 nm to 740 nm. In some embodiments, photosensitizers are activated and become cytotoxic at a wavelength of about 600 nm, 610 nm, 620 nm, 630 nm, 640 nm, 650 nm, 660 nm, 670 nm, 680 nm, 681 nm, 682 nm, 683 nm, 684 nm, 685 nm, 686 nm, 687 nm, 687 nm, 689 nm, 690 nm, 700 nm, 710 nm, 720 nm, 730 nm, 740 nm, 750 nm, 760 nm, 770 nm, 780 nm, 790 nm or 800 nm. In some embodiments, photosensitizers are activated at a wavelength of less than 600 nm or more than 800 nm. In some embodiments, photosensitizers are activated at a wavelength of 689 nm. Suitable wavelengths for photosensitive molecule activation will depend on the particular photosensitive molecule used.

In some embodiments, the methods comprise exposing a virus-related particle that includes a photosensitive molecule to infrared, near-infrared, or ultraviolet light. The energy delivered by the laser may range, for example, from about 25 Joules (J) to about 200 J, for example, about 25 J, about 50 J, about 75 J, about 100 J, about 125 J, about 150 J, about 175 J, or about 200J. In some embodiments, the cumulative fluence of exposure of the target urothelial tumor to a laser is about 50 J/cm² to about 200 J/cm², for example, about 50 J/cm², 75 J/cm², 100 J/cm², 150 J/cm², 175 J/cm², or 200 J/cm².

The amount of time a photosensitive molecule (e.g., a photosensitizer) is exposed to a particular wavelength of light can depend in part on the type of molecule and/or desired function and/or therapeutic effect resulting from the exposure. In some embodiments, a virus-related particle that includes a photosensitive molecule is exposed to an excitation wavelength of light for about 5 seconds to about 5 minutes. For example, a virus-related particle that includes a photosensitive molecule can be exposed to an excitation wavelength of light for about 5 to 60 seconds to activate the molecule. In some embodiments, a virus-related particle that includes a photosensitive molecule is exposed to an excitation wavelength of light for at least 1, 1.5, 2, 2.5, 3, 3.5, 4, 4.5 or 5 minutes, or more. In some embodiments, a virus-related particle that includes a photosensitive molecule is exposed to an excitation wavelength of light for at least 65 minutes or about 5 minutes. In some embodiments, a virus-related particle that includes a photosensitive molecule is exposed to an excitation wavelength of light for at least 6 minutes or about 6 minutes. In some embodiments, a virus-related particle that includes a photosensitive molecule is exposed to an excitation wavelength of light for at least 7 minutes or about 7 minutes. It should be understood that the length of time a therapeutic agent is exposed to an excitation wavelength can vary depending, for example, on the energy (e.g., wattage) of the laser. For example, lasers with a lower wattage may be applied to a therapeutic agent for a longer period of time in order to activate a photosensitive molecule.

In some embodiments, exposing the target tumor to a laser comprises advancing a cystoscope comprising a treatment laser into the bladder and positioning the treatment laser over the target tumor midpoint to produce a laser spot size of about 1 cm. In some embodiments, exposing the target tumor to a laser comprises advancing a cystoscope comprising a treatment laser into the bladder and positioning the treatment laser over the target tumor midpoint to produce a laser spot size of about 2 cm. In some embodiments, exposing the target tumor to a laser comprises advancing a cystoscope comprising a treatment laser into the bladder and positioning the treatment laser over the target tumor midpoint to produce a laser spot size of about 3 cm.

In some embodiments, exposing the target tumor to a laser comprises delivering a total light dose of about 50 J/cm². In some embodiments, exposing the target tumor to a laser comprises delivering a total light dose of about 75 J/cm². In some embodiments, exposing the target tumor to a laser comprises delivering a total light dose of about 100 J/cm². In some embodiments, exposing the target tumor to a laser comprises delivering a total light dose of about 125 J/cm². In some embodiments, exposing the target tumor to a laser comprises delivering a total light dose of about 150 J/cm².

In some embodiments, exposing the target tumor to a laser comprises: (a) advancing a cystoscope comprising a treatment laser into the bladder and positioning the treatment laser over the target tumor midpoint to produce a laser spot size of about 2 cm; and (b) delivering a total light dose of about 100 J/cm².

In some embodiments, a Modulight ML7710 Laser is used to deliver a treatment laser (modulight.com/ml7710).

In some embodiments, a method comprises advancing into the bladder a cystoscope comprising a working channel into which an open-ended flexitip ureteral catheter has been inserted. A cystoscope comprising a working channel is a medical endoscopic device designed for visualization and access to the interior of the bladder and urethra. It includes an integrated working channel, which is a dedicated lumen within the cystoscope that allows the passage of instruments such as needles, forceps, or catheters during diagnostic or therapeutic procedures. This channel enables one to perform interventions—such as injections, biopsies, or resection—under direct visual guidance. The working channel can vary in size (typically measured in French, Fr) and is typically compatible with both rigid and flexible cystoscope designs, depending on the clinical application. An open-ended flexitip ureteral catheter is a flexible medical device designed for navigation and access within the urinary tract, for example, the ureter. In some embodiments, it includes a soft, flexible distal tip—the “flexitip”—which facilitates atraumatic insertion and advancement, reducing the risk of injury to delicate urologic tissues, for example. The open-ended design means the distal tip of the catheter has an opening that allows for fluid drainage, contrast injection, and/or guidewire passage. This type of catheter is commonly used in conjunction with cystoscopes for drug delivery into the upper urinary tract or bladder, for example.

In some embodiments, a method comprises positioning the cystoscope over the tumor midpoint at a distance of about 3 cm, about 4 cm, or about 5 cm from the bladder wall. In some embodiments, a method comprises positioning the cystoscope over the tumor midpoint at a distance of about 4 cm from the bladder wall.

In some embodiments, a method comprises removing the catheter without moving the cystoscope.

In some embodiments, a method comprises inserting a light delivery catheter (e.g., comprising a 1 cm tip) into the working channel of the cystoscope such that the tip of the catheter is positioned outside the cystoscope away from (e.g., about 3 cm away from) the bladder wall to produce a laser spot (e.g., having a 2 cm (diameter) laser spot size). In some embodiments, a light delivery catheter comprises an aiming laser and a treatment laser.

In some embodiments, a method comprises targeting light at the target tumor using the aiming laser to set the target area. The aiming laser of a light delivery catheter can be used, for example, to precisely identify and set the target area of a tumor prior to therapeutic light activation. This low-power visible laser—often red or green—is emitted through the catheter's optical fiber and projected onto the tissue surface to provide a clear visual reference for alignment. By adjusting the position and orientation of the catheter under endoscopic or image-guided visualization, one can direct the beam to the exact tumor region intended for treatment. Once the target area is confirmed, the therapeutic laser (typically of a specific wavelength and power for photodynamic or thermal therapy) is activated to deliver the intended dose to the delineated tissue.

The treatment laser of a light delivery catheter, in some embodiments, is used to deliver energy (light) directly to a target tumor with precision. Once the catheter is positioned and the tumor site is confirmed—for example, using an aiming beam or endoscopic guidance—the treatment laser is activated to emit a high-power beam of a specific wavelength and irradiance. This laser light is transmitted through the optical fiber within the catheter and directed onto the tumor tissue, where it initiates a therapeutic effect such as photodynamic activation of a drug. The beam's power, duration, and spot size are carefully controlled to achieve the desired energy dose (fluence), ensuring effective tumor treatment while minimizing damage to surrounding healthy tissue.

In some embodiments, a method comprises activating the treatment laser within the target area to deliver a total light dose of about 50 J/cm². In some embodiments, a method comprises activating the treatment laser within the target area to deliver a total light dose of about 75 J/cm². In some embodiments, a method comprises activating the treatment laser within the target area to deliver a total light dose of about 100 J/cm². In some embodiments, a method comprises activating the treatment laser within the target area to deliver a total light dose of about 125 J/cm². In some embodiments, a method comprises activating the treatment laser within the target area to deliver a total light dose of about 150 J/cm².

In some embodiments, a method comprises: (a) advancing into the bladder a cystoscope comprising a working channel into which an open-ended flexitip ureteral catheter has been inserted; (b) positioning the cystoscope over the tumor midpoint at a distance of about 4 cm from the bladder wall; (c) removing the catheter without moving the cystoscope; (d) inserting a light delivery catheter comprising a 1 cm tip into the working channel of the cystoscope such that the 1 cm tip is positioned outside the cystoscope about 3 cm away from the bladder wall to produce a 2 cm laser spot size, wherein the light delivery catheter comprises an aiming laser and a treatment laser; (e) targeting light at the target tumor using the aiming laser to set the target area; and (f) activating the treatment laser within the target area to deliver a total light dose of about 100 J/cm².

In some embodiments the light delivery catheter is a Medlight FD1 Frontal Light Distributor (medlight.com/products/frontal-light-distributor).

Irradiance refers to the power of electromagnetic radiation (such as light) incident on a surface per unit area. It is typically measured in watts per square meter (W/m²). In some embodiments, a treatment laser is configured to deliver light at an c of about 500 mW/cm² to about 1000 mW/cm². In some embodiments, a treatment laser is configured to deliver light at an irradiance of about 500 mW/cm². In some embodiments, a treatment laser is configured to deliver light at an irradiance of about 600 mW/cm². In some embodiments, a treatment laser is configured to deliver light at an irradiance of about 700 mW/cm². In some embodiments, a treatment laser is configured to deliver light at an irradiance of about 800 mW/cm².

In some embodiments, light from the treatment laser is delivered to the target tumor for a duration of about 100 to about 200 seconds. In some embodiments, light from the treatment laser is delivered to the target tumor for a duration of about 140 to about 180 seconds. In some embodiments, light from the treatment laser is delivered to the target tumor for a duration of about 150 to about 170 seconds. In some embodiments, light from the treatment laser is delivered to the target tumor for a duration of about 160 to about 170 seconds. In some embodiments, light from the treatment laser is delivered to the target tumor for a duration of about 165 to about 170 seconds. In some embodiments, light from the treatment laser is delivered to the target tumor for a duration of about 166 seconds.

“Exposing a virus-related particle to an excitation of light.” “exposing a virus-like drug conjugate (e.g., bel-sar) to an excitation of light.” or “exposing the target tumor to a laser” encompasses applying a light source (e.g., treatment laser) to the anatomical location at which the pharmaceutical composition comprising the virus-related particle has been delivered, e.g., at the base and/or proximate to the base of a target tumor (e.g., target urothelial tumor).

In some embodiments, a cystoscope is used to guide a light source (e.g., laser) emitting an excitation wavelength of light (e.g., 600 nm to 800 nm) to the site at which or near which the virus-related particle is delivered.

In some embodiments, a method comprises delivering to the target urothelial tumor a first dose of the pharmaceutical composition and delivering to the target urothelial tumor a first dose of a laser (e.g., laser treatment) 1 hour to 36 hours, 1 hour to 12 hours, 2 hours to 36 hours, 2 hours to 36 hours, or 12 hours to 36 hours, after the first dose of the pharmaceutical composition is delivered to the target urothelial tumor. In some embodiments, a first dose of a laser (e.g., laser treatment) is delivered about 1 hour, about 2 hours, about 12 hours, about 24 hours, or about 36 hours after the first dose of the pharmaceutical composition is delivered to the target urothelial tumor. In some embodiments, a method comprises delivering to the target urothelial tumor a first dose of the pharmaceutical composition and delivering to the target urothelial tumor a first dose of a laser (e.g., laser treatment) within the same day (e.g., within 1 hour, within 2 hours, within 3 hours, or within 12 hours). Delivery of a first dose of a pharmaceutical composition (e.g., via a single or multiple injections) and deliver of a first dose of a laser (e.g., a laser treatment) may be referred to herein as a single cycle of treatment (e.g., “Cycle 1” or a “first cycle of therapy”).

The first dose, in some embodiments, is a dose of about 10 μg to about 400 μg. In some embodiments, the first dose is about 20 μg to about 400 μg, or about 50 μg to about 400 μg (e.g., about 100 μg or 200 μg). In some embodiments, the first dose is about 50, about 100, about 200, about 400, about 600, or about 800 μg. In some embodiments, the first dose is about 50 μg. In some embodiments, the first dose is about 100 μg. In some embodiments, the first dose is about 200 μg. In some embodiments, the first dose is about 400 μg. In some embodiments, the first dose is about 600 μg. In some embodiments, the first dose is about 800 μg.

In some embodiments, the total dose (of the first dose) is delivered via a single injection, while in other embodiments, the total dose (of the first dose) is delivered via multiple injection (e.g., at least two injections) into the base of the target urothelial tumor and/or proximate to the base of the target urothelial tumor. In some embodiments, a single injection delivers 50 μg of the viral-related particle (e.g., virus-like drug conjugate, such as bel-sar). In some embodiments, a single injection delivers 100 μg of the viral-related particle (e.g., virus-like drug conjugate). In some embodiments, a single injection delivers 150 μg of the viral-related particle (e.g., virus-like drug conjugate, such as bel-sar). In some embodiments, a single injection delivers 200 μg of the viral-related particle (e.g., virus-like drug conjugate, such as bel-sar). In some embodiments, a single injection delivers 400 μg of the viral-related particle (e.g., virus-like drug conjugate, such as bel-sar). In some embodiments, a single injection delivers 800 μg of the viral-related particle (e.g., virus-like drug conjugate, such as bel-sar). In some embodiments, the total dose (of the first dose) is delivered via multiple injections. In some embodiments, the total dose (of the first dose) is 200 μg of the viral-related particle (e.g., virus-like drug conjugate, such as bel-sar) delivered by 2 injections of 100 μg of the viral-related particle (e.g., virus-like drug conjugate, such as bel-sar) or delivered by 4 injections of 50 μg of the viral-related particle (e.g., virus-like drug conjugate, such as bel-sar). In some embodiments, the total dose (of the first dose) is 400 μg of the viral-related particle (e.g., virus-like drug conjugate, such as bel-sar) delivered by 2 injections of 200 μg of the viral-related particle (e.g., virus-like drug conjugate, such as bel-sar), delivered by 4 injections of 100 μg of the viral-related particle (e.g., virus-like drug conjugate, such as bel-sar), or delivered by 8 injections of 50 μg of the viral-related particle (e.g., virus-like drug conjugate, such as bel-sar). In some embodiments, the total dose (of the first dose) is 800 μg of the viral-related particle (e.g., virus-like drug conjugate, such as bel-sar) delivered by 2 injections of 400 μg of the viral-related particle (e.g., virus-like drug conjugate, such as bel-sar), delivered by 4 injections of 200 μg of the viral-related particle (e.g., virus-like drug conjugate, such as bel-sar), delivered by 8 injections of 100 μg of the viral-related particle (e.g., virus-like drug conjugate, such as bel-sar), or delivered by 16 injections of 50 μg of the viral-related particle (e.g., virus-like drug conjugate, such as bel-sar).

In some embodiments, a method further comprises delivering to the target urothelial tumor a second dose of the pharmaceutical composition and delivering to the target urothelial tumor a second dose of a laser (e.g., laser treatment) 2 hours to 12 hours, or 12 hours to 36 hours, after the second dose of the pharmaceutical composition is delivered to the target urothelial tumor. In some embodiments, a second dose of a laser (e.g., laser treatment) is delivered about 1 hour, about 2 hours, about 12 hours, about 24 hours, or about 36 hours after the second dose of the pharmaceutical composition is delivered to the target urothelial tumor. In some embodiments, the second dose of the pharmaceutical composition is delivered about 1-2 weeks after the first dose of the pharmaceutical composition is delivered to the target urothelial tumor. In some embodiments, the second dose of the pharmaceutical composition is delivered about 1 week after the first dose of the pharmaceutical composition is delivered to the target urothelial tumor. In some embodiments, the second dose of the pharmaceutical composition is delivered about 2 weeks after the first dose of the pharmaceutical composition is delivered to the target urothelial tumor. Delivery of a second dose of a pharmaceutical composition (e.g., via a single or multiple injections) and deliver of a second dose of a laser (e.g., laser treatment) may be referred to herein as a single cycle of treatment (e.g., “Cycle 2” or a “second cycle of therapy”).

The second dose, in some embodiments, is a dose of about 10 μg to about 800 μg. In some embodiments, the second dose is about 50 μg to about 800 μg, about 20 μg to about 400 μg, or about 50 μg to about 400 μg (e.g., about 100 μg or 200 μg). In some embodiments, the second dose is about 200, about 400, about 600, or about 800 μg. In some embodiments, the second dose is about 50, about 100, about 200, about 400, about 600, or about 800 μg. In some embodiments, the second dose is about 50 μg. In some embodiments, the second dose is about 100 μg. In some embodiments, the second dose is about 200 μg. In some embodiments, the second dose is about 400 μg. In some embodiments, the second dose is about 600 μg. In some embodiments, the second dose is about 800 μg.

In some embodiments, the total dose (of the second dose) is delivered via a single injection, while in other embodiments, the total dose (of the second dose) is delivered via multiple injection (e.g., at least two injections) into the base of the target urothelial tumor and/or proximate to the base of the target urothelial tumor. In some embodiments, a single injection (of the second dose) delivers 50 μg of the viral-related particle (e.g., virus-like drug conjugate, such as bel-sar). In some embodiments, a single injection (of the second dose) delivers 100 μg of the viral-related particle (e.g., virus-like drug conjugate, such as bel-sar). In some embodiments, a single injection (of the second dose) delivers 150 μg of the viral-related particle (e.g., virus-like drug conjugate, such as bel-sar). In some embodiments, a single injection (of the second dose) delivers 200 μg of the viral-related particle (e.g., virus-like drug conjugate, such as bel-sar). In some embodiments, a single injection (of the second dose) delivers 400 μg of the viral-related particle (e.g., virus-like drug conjugate, such as bel-sar). In some embodiments, a single injection (of the second dose) delivers 800 μg of the viral-related particle (e.g., virus-like drug conjugate, such as bel-sar). In some embodiments, the total dose (of the second dose) is delivered via multiple injections. In some embodiments, the total dose (of the second dose) is 200 μg of the viral-related particle (e.g., virus-like drug conjugate, such as bel-sar) delivered by 2 injections of 100 μg of the viral-related particle (e.g., virus-like drug conjugate, such as bel-sar) or delivered by 4 injections of 50 μg of the viral-related particle (e.g., virus-like drug conjugate, such as bel-sar). In some embodiments, the total dose (of the second dose) is 400 μg of the viral-related particle (e.g., virus-like drug conjugate, such as bel-sar) delivered by 2 injections of 200 μg of the viral-related particle (e.g., virus-like drug conjugate, such as bel-sar), delivered by 4 injections of 100 μg of the viral-related particle (e.g., virus-like drug conjugate, such as bel-sar), or delivered by 8 injections of 50 μg of the viral-related particle (e.g., virus-like drug conjugate, such as bel-sar). In some embodiments, the total dose (of the second dose) is 800 μg of the viral-related particle (e.g., virus-like drug conjugate, such as bel-sar) delivered by 2 injections of 400 μg of the viral-related particle (e.g., virus-like drug conjugate, such as bel-sar), delivered by 4 injections of 200 μg of the viral-related particle (e.g., virus-like drug conjugate, such as bel-sar), delivered by 8 injections of 100 μg of the viral-related particle (e.g., virus-like drug conjugate, such as bel-sar), or delivered by 16 injections of 50 μg of the viral-related particle (e.g., virus-like drug conjugate, such as bel-sar).

In some embodiments, a method comprises delivering to a target bladder tumor (e.g., bladder cancer lesion) a first 50 μg to 200 μg dose of a pharmaceutical composition comprising a virus-like drug conjugate comprising a virus-like particle conjugated to a photosensitizer, wherein the first dose is delivered via a single injection or via multiple injections, and delivering to the target bladder tumor (e.g., bladder cancer lesion) a first 50 J/cm² to 100 J/cm² dose of a laser (e.g., laser treatment) for no more than 10 minutes, for example, about 5-7 minutes (e.g., 6 minutes), wherein the first dose of the laser (e.g., laser treatment) is delivered 2-36 hours, optionally 2-12 hours, after the first dose of the pharmaceutical composition is delivered.

In some embodiments, a method comprises delivering to a target bladder tumor (e.g., bladder cancer lesion) a first 200 μg dose of a pharmaceutical composition comprising a virus-like drug conjugate comprising a virus-like particle conjugated to a photosensitizer (e.g., a photosensitive VDC, wherein the first 200 μg dose is delivered via a single injection or via multiple injections, and delivering to the target bladder tumor (e.g., bladder cancer lesion) a first 50 J/cm² to 100 J/cm² dose of a laser (e.g., laser treatment) for no more than 10 minutes, for example, about 5-7 minutes (e.g., 6 minutes), wherein the first dose of the laser (e.g., laser treatment) is delivered about 24 hours after the first dose of the pharmaceutical composition is delivered.

In some embodiments, a method comprises delivering to a target bladder tumor (e.g., bladder cancer lesion) a first 400 μg dose of a pharmaceutical composition comprising a virus-like drug conjugate comprising a virus-like particle conjugated to a photosensitizer (e.g., a photosensitive VDC), wherein the first 400 μg dose is delivered via a single injection or via multiple injections, and delivering to the target bladder tumor (e.g., bladder cancer lesion) a first 50 J/cm² to 100 J/cm² dose of a laser (e.g., laser treatment) for no more than 10 minutes, for example, about 5-7 minutes (e.g., 6 minutes), wherein the first dose of the laser (e.g., laser treatment) is delivered about 24 hours after the first dose of the pharmaceutical composition is delivered.

In some embodiments, a method comprises delivering to a target bladder tumor (e.g., bladder cancer lesion) a first 800 μg dose of a pharmaceutical composition comprising a virus-like drug conjugate comprising a virus-like particle conjugated to a photosensitizer (e.g., a photosensitive VDC), wherein the first 800 μg dose is delivered via a single injection or via multiple injections, and delivering to the target bladder tumor (e.g., bladder cancer lesion) a first 50 J/cm² to 100 J/cm² dose of a laser (e.g., laser treatment) for no more than 10 minutes, for example, about 5-7 minutes (e.g., 6 minutes), wherein the first dose of the laser (e.g., laser treatment) is delivered about 24 hours after the first dose of the pharmaceutical composition is delivered.

In some embodiments, a method comprises delivering to the target bladder tumor (e.g., bladder cancer lesion) a second 50 μg to 200 μg dose of the pharmaceutical composition comprising a virus-like drug conjugate comprising a virus-like particle conjugated to a photosensitizer (e.g., a photosensitive VDC), wherein the second dose of the pharmaceutical composition is delivered 1-2 weeks after the first dose of the pharmaceutical composition is delivered, and delivering to the target bladder tumor (e.g., bladder cancer lesion) a second 50 J/cm² to 100 J/cm² dose of a laser (e.g., laser treatment) for no more than 10 minutes, for example, about 5-7 minutes (e.g., 6 minutes), wherein the second dose of the laser (e.g., laser treatment) is delivered 2-36 hours, optionally 2-12 hours, after the second dose of the pharmaceutical composition is delivered.

In some embodiments, a method comprises delivering to the target bladder tumor (e.g., bladder cancer lesion) a second 200 μg dose of the pharmaceutical composition comprising a virus-like drug conjugate comprising a virus-like particle conjugated to a photosensitizer (e.g., a photosensitive VDC), wherein the second dose of the pharmaceutical composition is delivered about 2 weeks after the first dose of the pharmaceutical composition is delivered, and delivering to the target bladder tumor (e.g., bladder cancer lesion) a second 50 J/cm² to 100 J/cm² dose of a laser (e.g., laser treatment) for no more than 10 minutes, for example, about 5-7 minutes (e.g., 6 minutes), wherein the second dose of the laser (e.g., laser treatment) is delivered about 24 hours after the second dose of the pharmaceutical composition is delivered.

In some embodiments, a method comprises delivering to the target bladder tumor (e.g., bladder cancer lesion) a second 400 μg dose of the pharmaceutical composition comprising a virus-like drug conjugate comprising a virus-like particle conjugated to a photosensitizer (e.g., a photosensitive VDC), wherein the second dose of the pharmaceutical composition is delivered about 2 weeks after the first dose of the pharmaceutical composition is delivered, and delivering to the target bladder tumor (e.g., bladder cancer lesion) a second 50 J/cm² to 100 J/cm² dose of a laser (e.g., laser treatment) for no more than 10 minutes, for example, about 5-7 minutes (e.g., 6 minutes), wherein the second dose of the laser (e.g., laser treatment) is delivered about 24 hours after the second dose of the pharmaceutical composition is delivered.

In some embodiments, a method comprises delivering to the target bladder tumor (e.g., bladder cancer lesion) a second 800 μg dose of the pharmaceutical composition comprising a virus-like drug conjugate comprising a virus-like particle conjugated to a photosensitizer (e.g., a photosensitive VDC), wherein the second dose of the pharmaceutical composition is delivered about 2 weeks after the first dose of the pharmaceutical composition is delivered, and delivering to the target bladder tumor (e.g., bladder cancer lesion) a second 50 J/cm² to 100 J/cm² dose of a laser (e.g., laser treatment) for no more than 10 minutes, for example, about 5-7 minutes (e.g., 6 minutes), wherein the second dose of the laser (e.g., laser treatment) is delivered about 24 hours after the second dose of the pharmaceutical composition is delivered.

In some embodiments, “delivery” excludes intravesical delivery, which is a route of administration that permits a pharmaceutical composition to fill the urinary bladder as a means of exposing a target urothelial tumor. By contrast, “delivery” herein refers to targeted delivery directly to the base of a target urothelial tumor or proximate to the base of a target urothelial tumor.

### Laser Photoactivation System

In some embodiments, a laser photoactivation system is used to activate the drug component of a virus-like drug conjugate or other photosensitive pharmaceutical composition. The system can be based on a modular, table-top laser platform designed for flexible use in photodynamic and other light-based therapies. In some embodiments, it includes one or more laser diode modules housed within a console and a fiber optic delivery device connected to the console. The fiber optic cable is, in some embodiments, a sterile, single-use, commercially available, small-diameter fiber compatible with insertion through the working channel of a flexible or rigid cystoscope. Its distal end can feature, for example, an optic element that generates a uniform laser beam with the desired diameter and divergence.

The photoactivation system, in some embodiments, also includes a cystoscope with a working channel into which an open-ended flexitip ureteral catheter or light delivery catheter can be inserted and advanced into the bladder. A representative light delivery catheter can include, for example, a 1 cm tip, which, when extended approximately 3 cm from the cystoscope, can generate a 2 cm laser spot size on the bladder wall. The catheter can incorporate both an aiming for precise targeting and a treatment laser to deliver therapeutic irradiance at a defined wavelength. These integrated components allow for controlled intravesical activation of the pharmaceutical agent with spatial precision and minimal systemic exposure. By activating the drug only in targeted regions, the laser photoactivation platform minimizes systemic exposure and enhances therapeutic precision, making it particularly useful in treating localized bladder pathologies such as carcinoma in situ or other urothelial malignancies.

In some embodiments, a light delivery catheter is a Medlight FD1 Frontal Light Distributor (medlight.com/products/frontal-light-distributor). The Medlight FD1 Frontal Light Distributor is a specialized light delivery catheter designed for precise and uniform illumination in photodynamic therapy (PDT) and other light-based medical applications. It comprises a fused silica optical fiber with a 600 μm core diameter and a numerical aperture (NA) of 0.37, ensuring efficient light transmission. The fiber is equipped with a proximal SMA905 connector compatible with standard laser systems and a distal microlens tip that produces a sharply defined, homogeneous circular light spot.

With an overall diameter of 2 mm and a length of 4 meters, the FD1 is designed for endoscopic use, fitting through the working channels of standard flexible endoscopes. It operates effectively across a wavelength range of 480 to 800 nm, accommodating various photosensitizers used in PDT. The device delivers a beam with a full divergence angle of 34.7°, achieving spot diameters of approximately 0.6 mm at the tip. 7.6 mm at 10 mm distance, and 13.2 mm at 20 mm distance. The light distribution maintains a uniformity within +15%, ensuring consistent therapeutic exposure. The FD1 is supplied sterile for single-use applications and is intended to be disposed of as medical waste after use.

In some embodiments, a Modulight ML7710 Laser is used to deliver a treatment laser beam (modulight.com/ml7710). The Modulight ML7710 is a versatile, multichannel medical laser platform engineered for a broad spectrum of clinical and preclinical applications, including photodynamic therapy (PDT), photoimmunotherapy, photothermal therapy, fluorescence imaging, and surgical procedures. Its modular design allows configuration with up to eight individually addressable laser channels, each customizable across a wavelength range from ultraviolet (UV) to over 3000 nm, with output powers ranging from 0 to 15 W per channel. The ML7710 features an intuitive touchscreen interface, integrated aiming beams, and a smart internal calibration module to ensure precise and consistent light delivery. Safety and usability are enhanced through built-in fiber sensors, foot/hand switches, and safety interlocks. Optional functionalities include real-time light dosimetry monitoring, fluorescence measurement, and customized treatment workflows

In some embodiments, a treatment laser is configured to deliver light at an irradiance of about 400 mW/cm². In some embodiments, a treatment laser is configured to deliver light at an irradiance of about 500 mW/cm². In some embodiments, a treatment laser is configured to deliver light at an irradiance of about 600 mW/cm². In some embodiments, a treatment laser is configured to deliver light at an irradiance of about 700 mW/cm². In some embodiments, a treatment laser is configured to deliver light at an irradiance of about 800 mW/cm².

### Additional Embodiments

Additional embodiments are described in the numbered paragraphs below:
1. A method for treating a target urothelial tumor in a human subject in need thereof, the method comprising: injecting into the base of a target urothelial tumor and/or into a site proximate to the base of the target urothelial tumor a pharmaceutical composition comprising a virus-related particle in a therapeutically effective amount sufficient to treat the target urothelial tumor and induce a urothelial field effect.
2. The method of paragraph 1, wherein the virus-related particle comprises a virus-like drug conjugate.
3. The method of paragraph 2, wherein the virus-like drug conjugate comprises a photosensitizer.
4. The method of any preceding paragraph, wherein the urothelial field effect is without systemic exposure to the virus-related particle and/or without local or system exposure of an immune checkpoint inhibitor.
5. The method of any preceding paragraph, wherein the urothelial field effect is within 7-14 days of injecting the pharmaceutical composition.
6. The method of any preceding paragraph, wherein the target urothelial tumor is malignant urothelial tumor.
7. The method of paragraph 6, wherein the malignant urothelial tumor is a urothelial carcinoma.
8. The method of any preceding paragraph, wherein the target urothelial tumor is in the bladder, and the urothelial field effect is in the bladder (i.e., a bladder urothelial field effect).
9. The method of paragraph 8, wherein the target urothelial tumor is a non-muscle invasive bladder cancer (NMIBC).
10. The method of any preceding paragraph, wherein the pharmaceutical composition is delivered below or around the base of the target urothelial tumor.
11. The method of paragraph 10, wherein the pharmaceutical composition is delivered above the muscularis propria of the bladder.
12. The method of any preceding paragraph, wherein injecting the pharmaceutical composition is via a single injection or multiple injections.
13. The method of any preceding paragraph, wherein the therapeutically effective amount is a total dose of about 50 μg to about 800 μg, about 50 μg to about 400 μg, or about 50 μg to about 200 μg.
14. The method of paragraph 13, wherein the total dose is delivered via at least two injections.
15. The method of paragraph 14, wherein each injection delivers about 50 μg to about 200 μg.
16. The method of any preceding paragraph, wherein:
   the concentration of the virus-related particle in each dose of the pharmaceutical composition is about 0.1 to about 1.0 mg/ml, optionally about 0.2 to about 0.6 mg/ml, and further optionally about 0.4 mg/ml; and/or
   the volume of each dose of the pharmaceutical composition is about 250 μl to about 2 ml; and/or
   the rate of deliver of the pharmaceutical composition is about 1-2 cc/min.
17. The method of any preceding paragraph further comprising exposing the target urothelial tumor to a laser.
18. The method of paragraph 17, wherein the cumulative fluence of exposure of the target urothelial tumor to the laser is about 50 J/cm² to about 200 J/cm², optionally about 75 J/cm² or about 100 J/cm².
19. The method of any preceding paragraph comprising:
   injecting into the target urothelial tumor a first dose of the pharmaceutical composition, wherein the first dose is delivered via a single injection or via multiple injections; and
   injecting into the target urothelial tumor a first dose of a laser within 36 hours, optionally 2 hours to 12 hours, after the first dose of the pharmaceutical composition is delivered to the target urothelial tumor.
20. The method of paragraph 19, wherein the first dose is 50 μg to 400 μg.
21. The method of paragraph 20, wherein the first dose is 100 μg or 200 μg.
22. The method of any one of paragraphs 19-21, further comprising:
   injecting into the target urothelial tumor a second dose of the pharmaceutical composition, wherein the second dose is delivered via a single injection or via multiple injections, and wherein the second dose is delivered about 1-2 weeks after the first dose of the pharmaceutical composition is delivered to the target urothelial tumor; and
   injecting into the target urothelial tumor a second dose of a laser 2 hours to 36 hours, optionally 2-12 hours, after the second dose of the pharmaceutical composition is delivered to the target urothelial tumor.
23. The method of paragraph 22, wherein the second dose is 50 μg to 400 μg.
24. The method of paragraph 23, wherein the second dose is 100 μg or 200 μg.
25. The method of any preceding paragraph, wherein the virus-like drug conjugate comprises a human papillomavirus (HPV) virus-like particle (VLP) comprising HPV L1 and/or L2 capsid proteins.
26. The method of any one of paragraphs 3-24, wherein the photosensitizer of the virus-like drug conjugate is an infrared dye.
27. The method of paragraph 26, wherein the infrared dye comprises a compound of Formula I:

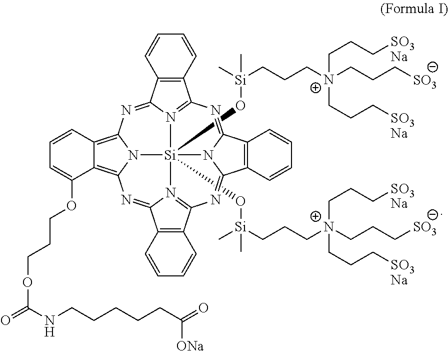

28. The method of paragraph 26, wherein the infrared dye comprises a compound of Formula II:

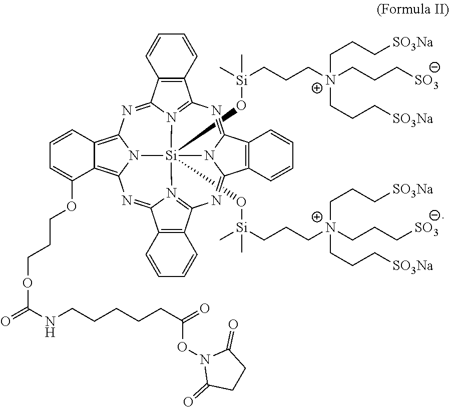

29. The method of any one of paragraph 17-28, wherein the wavelength of the laser is about 600 to about 800 nm, optionally about 689 nm.
30. The method of any preceding paragraph, wherein the human subject is an adult.
31. The method of any preceding paragraph, comprising:
   injecting into a target bladder tumor a first 50 μg to 400 μg dose of a pharmaceutical composition comprising a virus-like drug conjugate comprising a virus-like particle conjugated to a photosensitizer, wherein the first dose is delivered via a single injection or via multiple injections; and
   injecting into the target bladder tumor a first 50 J/cm² to 100 J/cm² dose of a laser for about 5-7 minutes, wherein the first dose of the laser is delivered within 36 hours, optionally 2-12 hours, after the first dose of the pharmaceutical composition is delivered.
32. The method of paragraph 31 further comprising:
   injecting into the target bladder tumor a second 50 μg to 400 μg dose of the pharmaceutical composition, wherein the second dose of the pharmaceutical composition is delivered 1-2 weeks after the first dose of the pharmaceutical composition is delivered; and
   injecting into the target bladder tumor a second 50 J/cm² to 100 J/cm² dose of a laser for about 5-7 minutes, wherein the second dose of the laser is delivered within 36 hours, optionally 2-12 hours, after the second dose of the pharmaceutical composition is delivered.
33. The method of paragraph 31 or 32, wherein the first dose and/or the second dose of the pharmaceutical composition are delivered into the base of the target bladder tumor.
34. The method of paragraph 31 or 32, wherein the first dose and/or the second dose of the pharmaceutical composition are delivered below the target bladder tumor and above the muscularis propria of the bladder.
35. The method of any one of paragraphs 31-34, wherein the virus-like drug conjugate comprises a virus-like particle comprising human papillomavirus (HPV) L1 and L2 capsid proteins conjugated to the photosensitizer.
36. The method of paragraph 35, wherein the virus-like particle is about 55 nm in diameter and comprises about 72 capsomeres, wherein each capsomere comprises about five HPV L1 capsid proteins and about one HPV L2 capsid protein.
37. The method of paragraph 36, wherein the HPV L1 capsid protein comprises the amino acid sequence of SEQ ID NO: 1.
38. The method of paragraph 36 or 37, wherein the HPV L2 capsid protein comprises the amino acid sequence of SEQ ID NO: 2.
39. The method of any one of paragraphs 32-38, wherein the virus-like drug conjugate comprises about 200 molecules of the photosensitizer conjugated to the HPV L1 and L2 capsid proteins of the virus-like drug conjugate.
40. The method of paragraph 39, wherein the laser has a wavelength of 689 nm, and the photosensitizer comprises the compound of Formula I:

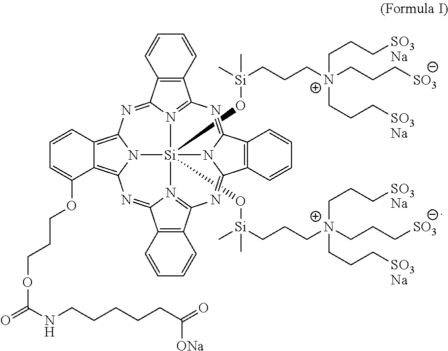

41. The method of paragraph 39 or 40, wherein the laser has a wavelength of 689 nm, and the photosensitizer comprises the compound of Formula II:

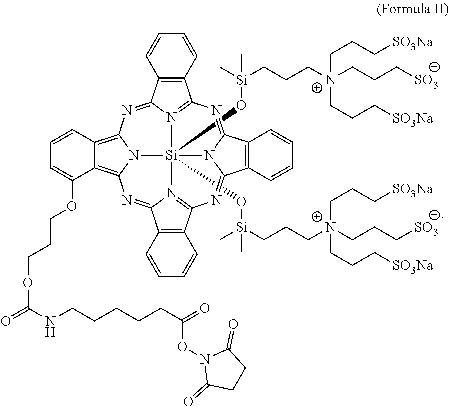

42. The method of any one of paragraphs 32-41, wherein the target bladder tumor is a non-muscle invasive bladder cancer.
43. The method of any one of paragraphs 32-42, wherein the method induces a bladder urothelial field effect without systemic exposure to the virus-related particle and/or without local or system exposure of an immune checkpoint inhibitor.
44. The method of any one of paragraphs 32-43, wherein the bladder urothelial field effect is within 7-14 days of injecting the pharmaceutical composition to the target bladder tumor.

### EXAMPLES

### Example 1. Phase 1 Clinical Trial

### Summary Overview

This Example describes positive interim results from an ongoing Phase 1 clinical trial of bel-sar (AU-011) in patients with non-muscle-invasive bladder cancer (NMIBC). Bel-sar is a virus-like particle conjugated to a light-activatable drug payload designed to have a dual mechanism of action. It induces direct tumor cell necrosis and pro-immunogenic cell death that elicits a robust and durable anti-tumor immune response. To date, the study has included 13 patients to evaluate the safety and feasibility of local administration of bel-sar alone (n=5) and bel-sar with light activation (n=8) with secondary endpoints to evaluate biological activity and immune mediated changes in the tumor microenvironment (TME). Ten of 13 study participants had low grade disease, which approximates the 70% incidence of this patient population among all NMIBC patients. Three of 13 study participants had high grade disease. In patients receiving bel-sar with light activation (n=8), four out of five patients with low grade disease demonstrated a clinical complete response with no tumor cells remaining on histopathological evaluation and two out of three patients with high grade disease demonstrated visual tumor shrinkage observed on cystoscopy. This treatment has demonstrated a rapid tumor response accompanied with a marked CD-8 T-cell infiltration observed in just a matter of days with a single low dose, which has the potential to translate into a durable response as an immune ablative treatment.

### Trial Design:

The ongoing Phase 1 trial is a two-part open-label clinical trial, designed to assess the safety and feasibility of bel-sar as a monotherapy. The treatment is administered seven to 12 days before the scheduled transurethral resection of bladder tumor (TURBT), the standard of care procedure. The participants are followed for safety monitoring over a 56-day period. The study is also evaluating bel-sar's biological activity with histopathological evaluation of tissue samples collected at the time of TURBT (regardless of tumor response) with evaluation of focal necrosis and immune changes in the tumor microenvironment. Part 1 of the trial is completed and involved five patients with low-grade NMIBC receiving a single bel-sar dose without light activation. Part 2 of the trial is ongoing and is expected to treat ten NMIBC patients, eight of which have been treated with 100 ug or 200 ug of bel-sar as a single dose as of the interim data cutoff date. Of these eight patients, five patients had low grade disease, and three patients had high grade disease. Seven out of these eight patients had a history of recurrent cancer and had undergone multiple TURBTs and adjuvant treatments such as Bacillus Calmette-Guerin (BCG), mitomycin, gemcitabine, cetrelimab, and tamoxifen prior to trial enrollment. In the planned expansion of this Phase 1 study, additional doses and treatment regimens will be tested.

### Safety Data:

In all 13 patients treated with bel-sar through the cut-off date, bel-sar was well-tolerated, with less than 10% of patients with Grade 1 treatment-emergent adverse events (TEAEs) related to study drug reported and no Grade 2 or higher study drug-related adverse events reported. No serious adverse events have been reported. No significant differences between the light-activated and non-light activated cohorts have been observed.

### Biological Activity:

The interim data in all eight patients receiving bel-sar with light activation showed clinical activity detectable seven to 12 days after a single low dose of bel-sar with light activation. This was demonstrated by histopathological evidence of complete response, necrosis, immune activation, or visual tumor shrinkage observed on cystoscopy. Of the patients with low-grade disease, four out of five exhibited a clinical complete response, with no tumor cells detected in histopathological evaluation post-treatment in the target and in several non-target tumors. Two of three of the patients with high grade disease demonstrated visual tumor shrinkage observed on cystoscopy. Immune activation was noted in all patients in both treated and untreated tumors with significant infiltration of effector CD8+ and CD4+ T cells (where immune staining was available). These data provide evidence of an unexpected bladder urothelial field effect with a single low dose of bel-sar with light activation, indicating a broader immune response in the bladder beyond the target tumor in these patients, without system exposure to bel-sar and without local or system exposure to an immune checkpoint inhibitor.

### Summary of Phase 1 Interim Results

Bel-sar demonstrates a favorable safety profile with robust clinical and immunological response in ‘all-comers’ NMIBC patients.
Highly favorable safety profile
   Focal treatment with no systemic adverse events
   Less than 10% drug-related adverse events
   No drug-related grade 2/3 adverse events, serious adverse events, or dose-limiting toxicities.
Robust immune activation
   Immune-mediated mode of action and bladder urothelial field effect
   100% of patients show immune cell infiltration in target and non-target lesions
Tumor shrinkage and clinical response
   Single low-dose of bel-sar demonstrates multiple complete responses in target and non-target tumors
   Positive interim results show 4/5 patients with low-grade disease had a complete clinical response
      Cohort 4a+4b: Single-Dose Drug with Light Activation (n=7)
Safety summary: no serious adverse events; no dose limiting toxicities
Strong safety profile: <10% of patients experienced Grade ≥1 TEAEs related to study drug; no Grade 2/3 adverse events related to study drug

| | | |
|---|---|---|
| Event | Grade | Number of patients |

Adverse events (related to study drug)

| | | |
|---|---|---|
| Nocturia | 1 | 1/7 |
| Urinary urgency | 1 | 1/7 |

Adverse events (related to injection or laser procedure)

| | | |
|---|---|---|
| Hematuria | 1 | 1/7 |
| Urinary blood clots | 1 | 1/7 |
| Nocturia | 1 | 1/7 |
| Urinary urgency | 1 | 1/7 |

Efficacy summary: Ta low-grade
4/5 low-grade target tumors demonstrate complete response to Bel-sar

| | Patient A1 | Patient A3 | Patient A4 | Patient B2 | Patient C1^{c} |
|---|---|---|---|---|---|
| Screening diagnosis | Single | Multiple | Multiple | Multiple | Multiple |
| | Ta low-grade | Ta low-grade | Ta low-grade | Ta low-grade | Ta low-grade |
| | | | Ta high-grade | | |
| AUA risk | Intermediate | Intermediate | Intermediate | Intermediate | Intermediate |
| classification | | | | | |
| Bel-sar dose/delivery | 100 μg | 100 μg | 100 μg | 100 μg | 200 μg |
| | IT/IM | IT/IM | IT/IM | IT | IT |
| Complete response: | ✓ | ✓ | ✓ | — | ✓ |
| Target tumor^{a} | | | | | |
| Complete response: | 2/2 | ½ | 1/1 | — | — |
| Non-target tumor^{a} | | | | | |
| (bladder urothelial | | | | | |
| field effect^{b}) | | | | | |
| Immune response: | ✓ | ✓ | ✓ | ✓ | pending |
| Target tumor | | | | | |
| Immune response: | ✓ | ✓ | ✓ | ✓ | pending |
| Non-target tumor | | | | | |
| Necrosis | ✓ | ✓ | ✓ | — | pending |
| Visual changes on | ✓ | ✓ | — | ✓ | ✓ |
| cystoscopy | | | | | |

^{a}Complete response defined as absence of tumor cells on histopathologic evaluation.
^{b}Bladder urothelial field effect: absence of tumor cells in non-target lesions.
^{c}Complete response (target tumor) based upon local pathology with central review ongoing; immune response and necrosis evaluations pending central review.
AUA, American Urological Association;
IM, intramural;
IT, intratumoral

Interim data demonstrated that single-dose (e.g., 100 μg) bel-sar generates innate and adaptive effectors regardless of immune environment, converting cold tumors into hot tumors (positive staining for CD45⁺/CD56⁺ Natural Killer (NK) cells, and positive staining for memory CD4⁺ T cells), producing memory T-cells, and driving tertiary lymphoid structure (TLS) formation (histological images not shown). 100% of treated and non-treated lesions were robustly infiltrated by immune cells; dendritic cells, neutrophils, and eosinophils were strongly associated with clinical response; in treated lesions, NK cell density unexpectedly increased up to 40x, and in treated lesions, CD4⁺ cytolytic T-cell density unexpectedly increased up to 7x, relative to pre-treatment lesions. In 5/5 participants, CD4⁺ and CD8+ memory T cells were observed after virus-like drug conjugate treatment.

### Light-Activated Cohorts (A+B):

Strong evidence of immune-mediated mechanism of action.
Bel-sar demonstrates evidence of producing pro-immunogenic changes in situ that bridge, activate and enhance adaptive immunity, consistent with its expected MOA.
100% (7/7) of target tumors showed significant infiltration of effector CD8+ T and CD4+ cells, as early as 7 days after laser activation.
100% (7/7) of non-target tumors^{a} showed T cell infiltration, supportive of a bladder urothelial field effect.
Focal eosinophilic infiltration was observed in 57% (4/7) target tumors and in 14% (1/7) non-target tumors, supportive of a local innate immune response to tumor necrosis.
Generation of lymphoid follicles^{b} was observed in 71% (5/7) target tumors, supportive of a local adaptive immune response.
^{a}Patients for which biopsies were available.
^{b}Organized aggregates of immune cells.
MOA, mechanism of action
   Efficacy summary: Ta high-grade
   3/3 high-grade tumors demonstrate immune response to Bel-sar

| | Patient A2 | Patient B1 | Patient B3 |
|---|---|---|---|
| Screening diagnosis | Single | Multiple | Single |
| | Ta high-grade | Ta high-grade | Ta High-grade |
| Screening AUA | High | High | Intermediate |
| risk classification | | | |
| Bel-sar dose/delivery | 100 μg | 100 μg | 100 μg |
| | IT/IM | IT | IT |
| Complete response: | — | — | — |
| Target tumor^{a} | | | |
| Complete response: | N/A | — | N/A |
| Non-target tumor^{a} (bladder | | | |
| urothelial field effect^{b}) | | | |
| Immune response: Target tumor | ✓ | ✓ | ✓ |
| Immune response: Non-target tumor | N/A | ✓ | N/A |
| Necrosis | — | — | — |
| Visual changes on cystoscopy | Tumor visually | Tumor visually | — |
| | smaller | smaller | |

^{a}Complete response defined as absence of tumor cells on histopathologic evaluation.
^{b}Bladder urothelial field effect: absence of tumor cells in non-target lesions.
^{c}Complete response (target tumor) based upon local pathology with central review ongoing; immune response and necrosis evaluations pending central review.
AUA, American Urological Association;
IM, intramural;
IT, Intratumoral

### Phase 1 Clinical Trial Protocol Synopsis

A Phase 1, open-label trial of bel-sar (AU-011) to determine the feasibility and safety of intratumoral injection with or without intramural injection in subjects with bladder cancer

Number of Subjects (planned): Approximately 21 adult subjects with bladder cancer

### Objectives:

1. To assess the feasibility and safety/tolerability of bel-sar using IT and/or IM injection alone and with laser administration.
2. To assess the focal distribution of bel-sar in the bladder wall and tumor using IT and/or IM injection alone and with laser administration.
3. To assess focal tumor necrosis after treatment with bel-sar IT and/or IM injection with laser administration in NMIBC subjects undergoing TURBT or NMIBC and MIBC subjects undergoing cystectomy.
4. To assess immune response following treatment with bel-sar (IT and/or IM injection alone and with laser administration).
5. To assess the systemic pharmacokinetics (PK) of bel-sar, once an assay is available, in all cohorts.

### Trial Endpoints:

### Primary Endpoint:

Incidence and severity of treatment-related adverse events (AEs) and serious adverse events (SAEs) and incidence of dose-limiting toxicities (DLTs).

### Secondary Endpoints:

Successful IT and/or IM injection procedure: defined as a full dose being injected.
Focal distribution of bel-sar using immunohistochemical (IHC) staining.
Presence of focal necrosis in bladder tumor, based on histopathology in Cohorts 4 and 5.
Evidence of immune response, i.e., immune cell infiltration on histology in Cohorts 4 and 5 using immune markers.
Incidence of adverse device effects (ADEs) and serious adverse device effects (SADEs) with respect to the laser device.
Systemic bel-sar PK profile in all cohorts.

### Trial Design:

This is a “window of opportunity” trial in bladder cancer subjects who are planned to undergo TURBT or cystectomy per standard of care (SoC). The goal is to achieve the trial objectives with minimal disruption to the SoC of the treating Investigator. The trial will enroll approximately 21 subjects with NMIBC and MIBC into 6 cohorts (Cohort 1b, Cohorts 4a, 4b, & 4c, and Cohorts 5a & 5b).

Cohort 1b—Injection Phase with TURBT (Approximately 5 NMIBC Subjects):

This cohort is designed to evaluate the feasibility, safety, and distribution of bel-sar alone (50 μg intramural (IM) and 50 μg Intratumoral (IT)) followed by TURBT (need for procedure per SoC) approximately 24-48 hours post injection in subjects with NMIBC.

Cohort 4—Injection(s) and Laser Phase with TURBT (Approximately 10 NMIBC Subjects):

This cohort is designed to evaluate the feasibility, safety, distribution, and necrosis caused by bel-sar injection(s) (IT with or without IM injection) followed by laser administration.

Cohort 4a—Approximately 4 subjects will be enrolled and will receive bel-sar injections of 50 μg IT and 50 μg IM on Day 1, laser administration on Day 2, and TURBT on Day 9 (+1 day).

Cohort 4b—Approximately 3 subjects will receive bel-sar IT injection(s) of 100 μg. Subjects will undergo bel-sar injection(s) on Day 1, laser administration on Day 2, and TURBT on Day 14 (+7 days).

Cohort 4c—Approximately 3 subjects will receive bel-sar IT injection(s) of 200 μg. Subjects will undergo bel-sar injection(s) on Day 1, laser administration on Day 2, and TURBT on Day 14 (+7 days).

Cohort 5—Injection and Laser Phase with Cystectomy (Approximately 6 NMIBC or MIBC Subjects):

This cohort is designed to evaluate the feasibility, safety, distribution, and necrosis caused by bel-sar IT injection(s) followed by laser administration.

Cohort 5a—Approximately 3 subjects will undergo bel-sar IT injection(s) of 100 μg on Day 1, laser administration on Day 2, and cystectomy on Day 14 (+7 days).

Cohort 5b—Approximately 3 subjects will undergo bel-sar IT injection(s) of 200 μg on Day 1, laser administration on Day 2, and cystectomy on Day 14 (+7 days).

IP Administration by Tumor Size (Cohorts 5a and
5b) Subjects receiving 100 OR 200 μg bel-sar IT

| bel-sar | | | |
|---|---|---|---|
| Total | Tumor | | |
| Dose | Size | | bel-sar Injection(s) |
| 100 μg | <2 | cm | 100 μg IT (at the base of tumor) |
| 100 μg | ≥2 | cm | 50 μg IT (at the base of tumor) |
| | | | 50 μg IT (at the base of tumor) |
| | | | Inject at opposite sides of the tumor |
| | | | base (if technically feasible). |
| 200μ | <2 | cm | 200 μg IT (at the base of tumor) |
| 200μ | ≥2 | cm | 100 μg IT (at the base of tumor) |
| | | | 100 μg IT (at the base of tumor) |
| | | | Inject at opposite sides of the tumor |
| | | | base (if technically feasible). |

### Trial Methods:

Subjects will be screened and enrolled based upon eligibility criteria and after providing written informed consent between Day −28 and Day −1. Screening procedures include medical history, surgical history, urologic assessments, risk assessments, vital signs, and safety laboratory analyses. To be eligible for enrollment, subjects must have a diagnosis of urothelial carcinoma (Refer to Inclusion/Exclusion Criteria for additional details).

Once all eligibility criteria are confirmed, subjects will be assigned to a Cohort based on available openings and planned SoC procedure (cystectomy or TURBT). Subjects will receive treatment and be followed for at least 56 days with the following assessments collected as specified by Cohort in the SoA:
Treatment emergent and treatment related AEs.
Concomitant medications.
Medical history/Surgical history.
Risk assessment and NMIBC/MIBC status.
Hematology and serum chemistry (complete blood count with differential and Comprehensive Metabolic Panel).
Blood samples for PK analysis, including samples for population PK.
Anti-drug antibodies (ADA) samples for ADA analysis.
Urinalysis.
Electrocardiogram.
Physical Examination (PE).
Eastern Cooperative Oncology Group (ECOG) Performance Status Scale.
Vital signs (blood pressure, temperature, pulse, and respiration) including height and weight (body mass index will be calculated).
Flexible or rigid cystoscopy with bel-sar injection(s) (all cohorts).
Biopsies:
   Biopsy of the target tumor at a location away from the site of injection.
   Biopsy of a non-target tumor, if feasible.
   Biopsy to confirm urothelial carcinoma (if needed).
Flexible or rigid cystoscopy with laser administration (Cohorts 4 and 5).
Cystoscopy with TURBT (Cohorts 1b and 4).
Cystectomy (Cohort 5 only).
Digital video or photography of the injection procedure (when available).

### Inclusion Criteria:

Subjects must:
1. Be at least 18 years of age.
2. Have been informed about the investigative nature and requirements of the trial, voluntarily agreed to participate in the trial and follow all trial procedures and have documented their consent by signing the Informed Consent Form before participating in any trial-related activities.
3. Subjects must have urothelial carcinoma of the bladder.
   Note: For subjects with recurrence of prior NMIBC, have a current lesion that clinically appears to be NMIBC (per the Investigator's judgement based on cystoscopy) and was confirmed histologically (within the last 12 months).
   Note: For subjects with first time NMIBC, have confirmation of urothelial carcinoma by recent biopsy (≤6 months of Screening Visit) and the disease must clinically appear as NMIBC based on Investigator judgement.
   Note: For Cohort 5, MIBC is allowed but must be demonstrated pathologically. Subjects must have residual MIBC of ≥1 cm per investigator's clinical assessment.
4. Have no evidence of metastatic disease confirmed by abdominal and pelvic imaging (e.g. computerized tomography [CT] scan, magnetic resonance imaging, positron emission tomography scan, or CT urogram [for subjects without a contrast allergy or a history of chronic kidney disease]).
   Note: Only bladder tumors may be treated with bel-sar. Upper tract or prostatic urethra lesions cannot be the lesion injected in this trial, but may be present (i.e., Investigator can treat upper tract or prostatic urethra per SoC as long as this treatment will not interfere with the local bel-sar injection of the trial tumor).
5. Have a clinical diagnosis of NMIBC or histopathological diagnosis of MIBC:
   a. Cohorts 1b and Cohort 4 require TURBT per Investigator's SoC and judgement.
   b. Cohort 5 requires cystectomy per Investigator's SoC and judgment (i.e., high risk NMIBC with BCG failure, or carcinoma in situ, or subjects with MIBC who are not eligible to receive neoadjuvant chemotherapy per Investigator's SoC).
   c. One of the tumors (papillary or carcinoma in situ lesions) must be ≥1.0 cm in greatest dimension and must be accessible for bel-sar injection per Investigator judgement.
6. If female and capable of becoming pregnant, agree to have pregnancy testing performed at screening (must be negative) and if required within 24 hours of bel-sar injection (must be negative); must agree to use highly effective method of contraception from the date of the screening pregnancy test; and must agree to continue using such precautions for 90 days after the final dose of bel-sar. Women considered capable of becoming pregnant include all females who have experienced menarche and have not experienced menopause (as defined by amenorrhea for greater than 12 consecutive months) or have not undergone successful surgical sterilization (hysterectomy, bilateral tubal ligation, or bilateral oophorectomy).
7. If male, must be of non-reproductive potential (i.e., have azoospermia either from a vasectomy or from an underlying medical condition) or must use a highly effective method of contraception defined as one that results in a low failure rate (i.e., less than 1% per year) when used consistently and correctly. Males with sexual partners of childbearing potential must agree to use a highly effective form of barrier birth control as outlined in the protocol or abstain from sexual intercourse during the trial and for 90 days after a bel-sar injection. Males should not donate sperm until 90 days after injection of bel-sar.
8. Be able and willing to avoid all prohibited medications for the appropriate washout period and during trial follow up.
9. Adequate bone marrow, renal, and hepatic function as defined by:

Adequate Organ Function Definitions

| | |
|---|---|
| System | Laboratory Values |

| Hematologic | | |
|---|---|---|
| Absolute Neutrophil Count | >1200 | cells/μL |
| Hemoglobin | >9 | g/dL |
| Platelets | >75,000/μL | |
| Hepatic | | |
| ALT and AST | ≤3.0 × ULN | |
| Total bilirubin | ≤1.5 × ULN unless the subject has | |
| | documented/suspected | |
| | Gilbert's, then ≤3 × ULN | |
| Albumin | ≥3.0 g/dL (30 g/L) | |
| Renal | | |
| Glomerular Filtration Rate | ≥30 | mL/min |

ALT: alanine aminotransferase; AST: aspartate aminotransferase ULN: upper limit of normal.

### Exclusion Criteria:

### Subjects:

1. Must not have current or history of metastatic urothelial carcinoma.
2. Must not have any additional malignancy that requires active treatment. Exceptions include:
   a. Basal cell carcinoma of the skin or squamous cell carcinoma of the skin that has undergone potentially curative therapy with evidence of remission for at least 1 year.
   b. In situ cervical cancer treated and with at least 1 year without recurrence.
   c. Any other subject felt appropriate by the Investigator upon discussion with trial's Medical Monitor.
3. Must not have any contraindication to general or spinal anesthesia.
4. Is unable to undergo flexible or rigid cystoscopy.
5. Must not be pregnant or lactating.
6. Must not have used an investigational drug or medical device within 30 days or 5 half-lives (whichever is longer) of Visit 1 or concurrent enrollment in another investigational trial.
7. Must not have known contraindications or sensitivities to phthalocyanine-based dye, bel-sar, the capsid component, or prior treatment with laser.
8. Must not have active autoimmune disease, chronic inflammatory condition, conditions (like solid organ transplant or bone marrow allograft) requiring concurrent use of any systemic immunosuppressants or steroids.
9. Must not have a known immune-deficiency state(s)-including, but not limited to HIV, myelodysplastic disorders, marrow failures, history of solid organ transplant, or bone marrow allograft.
10. Must not be receiving continuous anticoagulation or antiplatelet therapy; temporary discontinuation of anticoagulation or antiplatelet therapy for tumor biopsies and/or injection to be safely performed should be managed per local standards and discussed with the Medical Monitor.
11. Must not have received any systemic anticancer therapy not otherwise mentioned above within the last 28 days.
12. Must not have active bacterial, fungal, or viral infections-all prior infections must have resolved following optimal therapy, and subjects must be off all systemic anti-infective agents.
13. Must not have a history of coagulopathy resulting in uncontrolled bleeding, e.g., hemophilia, von Willebrand's disease, etc.
14. Must not have a history of human immunodeficiency virus or chronic active hepatitis B or C.
15. Cardiac Exclusions:
   a. New York Heart Association Class 3 or 4 congestive heart failure.
   b. Uncontrolled hypertension.
   c. Unstable angina pectoris.
   d. Clinically important cardiac arrhythmia (e.g., requiring anti-arrhythmic drugs like sotolol).
   e. Mean QT interval corrected for heart rate (QTc)≥470 ms calculated from an ECG using Fredericia's Correction by manual read.
16. Must not have any other condition or previous surgery that, in the opinion of the Investigator (with Sponsor input as needed), would interfere with trial participation, evaluation of the investigational product, interpretation of trial results, or put the subject at any unnecessary risk.

### Investigational Product, Dosage, and Mode of Administration:

The investigational treatment includes a drug (bel-sar), and a laser (Laser Photoactivation System) that delivers 689 nm light to activate the drug once it is bound to the tumor cells.

Bel-sar is an isotonic solution comprising a recombinantly manufactured virus-like drug conjugate comprising (a) an empty proteinaceous virus-like particle (VLP) having a diameter of about 55 nm and including about 72 capsomeres, wherein each capsomere includes about five modified HPV L1 capsid proteins (SEQ ID NO: 1) and about one HPV L2 capsid protein (SEQ ID NO: 2); and (b) about 200 molecules of a phthalocyanine-based dye of Formula I (IRDye® 700DX; “Dye”) conjugated to the capsid proteins of the VLP. The VLP and Dye combination is a virus-like drug conjugate. Bel-sar drug product is a sterile, pale blue aqueous solution supplied at a concentration of 0.4 mg per mL of buffered isotonic solution (pH 6.5). Bel-sar is supplied in a 2.0 mL, single-use vial; each vial contains a fill volume of 0.4 mL.

Drug is administered via IT injection with or without IM injection using a cystoscopic approach.

For all cohorts except 1b, the Laser Photoactivation System consists of a laser console and a frontal light distributor. The laser light (689 nm) is applied to the bladder tumors.

| Cohort | Bel-sar Injection (Day 1) | Laser Administration | Surgery |
|---|---|---|---|
| Cohort 1b | 50 μg in 250 μL IT (at the base of the | Not applicable | TURBT within 24 hours |
| 5 NMIBC | tumor above the area of lamina propria | | after bel-sar injection |
| subjects | injection) | | |
| | 50 μg in 250 μL IM (at the tumor edge in | | |
| | lamina propria) | | |
| Cohort 4a | 50 μg in 250 μL IT (at the base of the | Administered Day 2 | TURBT Day 9 ± 1 D |
| Approximately | tumor above the area of lamina propria | | |
| 4 NMIBC | injection) | | |
| Subjects | 50 μg in 250 μL IM (at the tumor | | |
| | edge in lamina propria) | | |
| Cohort 4b | 100 μg IT (at the base of the tumor)^{a} | Administered Day 2 | TURBT Day 14 ± 7 D |
| Approximately | | | |
| 3 NMIBC | | | |
| Subjects | | | |
| Cohort 4c | 200 μg IT (at the base of the tumor)^{a} | Administered Day 2 | TURBT Day 14 ± 7 D |
| Approximately | | | |
| 3 NMIBC | | | |
| Subjects | | | |
| Cohort 5a | 100 μg IT (at the base of the tumor)^{a} | Administered Day 2 | TURBT Day 14 ± 7 D |
| Approximately | | | |
| 3 NMIBC OR | | | |
| MIBC subjects | | | |
| Cohort 5b | 200 μg IT (at the base of the tumor)^{a} | Administered Day 2 | TURBT Day 14 ± 7 D |
| Approximately | | | |
| 3 NMIBC OR | | | |
| MIBC subjects | | | |

TURBT: transurethral resection of the bladder tumor; MIBC: muscle invasive bladder cancer; NMIBC non-muscle invasive bladder cancer; IT: intratumoral; IM: intramural.
^{a}Cohorts 4b and 4c and 5a and 5b: For subjects receiving the 100 μg or 200 μg IT dose with a target tumor ≥2 cm, the dose will be split into 2 equal IT injections administered at opposite sides of the base of the tumor (if technically feasible). For subjects with a target tumor <2 cm, a single injection will be administered at the specified dose.

### Feasibility Analyses:

The feasibility analyses will be descriptive in nature. The following endpoints will be summarized by cohort and overall:
The incidence of successful IM and IT injections.
Presence of focal necrosis in bladder tumor (in treated area) assessed using histopathology.
Systemic PK profile and ADA parameters will be summarized.
Within each cohort, the criteria for procedure feasibility are successful injections in ≥50% of the subjects, ≤1 moderate injection-related AE, and ≤1 DLT. Across all cohorts, the overall procedure feasibility criteria are successful injections in ≥75% of subjects, with ≤10% of subjects with moderate injection-related AEs, and ≤10% of subjects with DLTs.

### Sample Size:

The sample size of approximately 5 subjects in the Injection Phase (Cohort 1b) and approximately 16 subjects in the Injection and Laser Phase (approximately 10 in Cohort 4 and approximately 6 in Cohort 5) is empiric and based on clinical observation and decision.

### Example 2. Amended Phase 1 Clinical Trial

### Protocol Synopsis

### Objectives:

1. To assess the feasibility and safety/tolerability of bel-sar using IT and/or IM injection alone and with laser application (also referred to herein as “laser treatment”).
2. To assess the focal distribution of bel-sar in the bladder wall and tumor using IT and/or IM injection alone and with laser application.
3. To assess focal tumor necrosis after treatment with bel-sar IT and/or IM injection (alone and with laser application).
4. To assess immune response following treatment with bel-sar (IT and/or IM injection alone and with laser application).
5. To assess the systemic pharmacokinetics (PK) of bel-sar.
6. To assess preliminary efficacy of bel-sar.

### Trial Endpoints:

### Primary Endpoint:

Incidence and severity of treatment-related adverse events (AEs) and serious adverse events (SAEs) and incidence of dose-limiting toxicities (DLTs).

### Secondary Endpoints:

Successful IT and/or IM injection procedure: defined as a full dose being injected.
Focal distribution of bel-sar using immunohistochemical (IHC) staining.
Presence of focal necrosis in bladder tumor, based on histopathology in Cohorts 4a-g and the muscle invasive bladder cancer (MIBC) Cohort.
Immune response in all cohorts.
Incidence of adverse device effects (ADEs) and serious adverse device effects (SADEs).
Systemic bel-sar PK profile in all cohorts.
Complete response (CR) rate at the 3-month timepoint.
CR rate at any time.
Duration of Response (DOR): Length of time participants maintain a CR after achieving a CR (6-, 9-, and 12-month timepoints).
Durable CR Rate: Proportion of participants maintaining a CR after achieving a CR over a specified period (6-. 9-, and 12-month timepoints).

### Exploratory Endpoints:

Unstructured participant feedback regarding bel-sar treatment and schedule.
Urinary Biomarkers.

### Trial Design:

This is a Phase 1 trial enrolling participants with urothelial carcinoma to evaluate the safety, technical feasibility, and preliminary efficacy of bel-sar. The goal is to achieve the trial objectives with minimal disruption to the standard of care (SoC) of the treating Investigator.

Cohorts 1a, 2, 3, and 6 have been closed without enrollment, while Cohorts 1b, 4a, 4b, and 4c have completed enrollment. In the current amendment, the updated trial design reflects closure of Cohorts 5a-b, as well as the inclusion of additional Cohorts 4d-g and an MIBC cohort.

### Rationale for Continued Dose Escalation and Evaluation of Both Safety and Preliminary Efficacy

This trial was initially designed as a “window of opportunity” trial, in which non-muscle invasive bladder cancer (NMIBC) participants were administered single doses of bel-sar before their planned SoC transurethral resection of the bladder tumor (TURBT). Given the short follow-up between bel-sar treatment and TURBT (3 weeks or less in the cohorts enrolled thus far), the primary objective was to evaluate safety and technical feasibility, but not efficacy. However, encouraging preliminary drug activity from single doses of bel-sar was observed.

A total of 16 participants with NMIBC have been treated, of which 5 received bel-sar without light activation and 11 received bel-sar across 2 dose levels with light activation. Of the 11 participants who received drug with light activation, 7 had low-grade disease and 4 had high-grade disease. At the time of this amendment, efficacy data based upon central pathology review of TURBT specimens is available for 8 participants treated with bel-sar and light activation. These data show complete clinical response of the target lesion as assessed by histopathological evaluation in 4 out of 5 participants with low-grade target lesions, who received bel-sar with light activation between 7 to 12 (+7) days before their scheduled SoC TURBT per protocol.

Histopathologic CR was defined as absence of tumor cells in the TURBT specimen. Of note, 3 out of 5 of these participants with low-grade tumors also demonstrated histopathologic CR in several distant, non-target (non-treated) tumors at time of TURBT. Two of 3 participants with high-grade disease demonstrated visual tumor shrinkage at the time of TURBT, while tumor cells were still present on histopathologic evaluation. In all 8 participants with low or high-grade tumors receiving bel-sar with light activation, CD4+ and CD8+ T cell infiltration was visualized in all target (treated) and all non-target (non-treated) bladder tumors 7 to 12 (+7) days after injection. The detection of lymphoid follicles in 5 out of 7 target tumors further supports the generation of a local adaptive immune response, consistent with the expected mechanism of action of bel-sar.

Based on this positive preliminary evidence of drug activity, this Phase 1 trial is expanded to conduct further dose finding through continued dose escalation, as well as to explore different treatment schedules, with the goal of achieving durable clinical CR with bel-sar. In the current amendment, a longer follow-up period enables both the collection of additional safety data and a longitudinal assessment of efficacy that is clinically meaningful to facilitate progression to a pivotal trial. The expectation is that safety and response data obtained from this amendment will help identify a final dose and treatment schedule for future trials with registrational intent.

The trial will enroll up to 55 participants, comprised of approximately 34-40 participants with NMIBC (in Cohorts 1b and Cohorts 4a-j), approximately 3 participants with MIBC (in the MIBC cohort), and with the potential to backfill up to 12 participants.

### Rationale for a Multiple Treatment Cycle Strategy: 2-Cycle and 3-Cycle Cohorts

Safety review of Cohorts 4a-4c demonstrated a single dose of bel-sar (up to 200 μg) was safely tolerated and technically feasible. No SAEs or DLTs were observed. Less than 10% of participants experienced Grade 1 treatment-emergent adverse events related to bel-sar and no Grade 2 or 3 AEs related to the bel-sar were observed. Peripheral whole blood samples collected for PK sampling post bel-sar injection demonstrated that systemic PK was below the limit of quantification in all samples tested. Therefore, bel-sar distribution was limited to the injection sites in the bladder, consistent with the lack of systemic AEs observed. Procedure feasibility, as defined in the protocol, considers both the success (full dose) of bel-sar injections and the incidence of injection-related AEs. Thus far, procedure feasibility was met across all cohorts enrolled (Cohorts 1b and 4a-c). Bel-sar is an immune-ablative therapy that induces pro-immunogenic cell death of tumor cells to generate local innate and adaptive effector immunity. Given the technical feasibility and favorable safety profile of bel-sar administered in single doses up to 200 μg, a “prime-boost” strategy similar to conventional vaccine approaches is proposed.

In the current amendment, the updated trial design reflects closure of Cohorts 5a-b, as well as the inclusion of additional Cohorts 4d-j in which participants will be administered multiple treatment cycles to augment the bel-sar driven immune response and potentially lengthen the durability of tumor response. Additionally, Cohorts 4f, 4g, and 4i will explore neoadjuvant regimen in which participants will be administered multiple treatment cycles to augment the bel-sar prior to undergoing TURBT. Cohorts 4h and 4i will evaluate an optional high dose with and without TURBT.

In the additional cohorts, each treatment cycle constitutes 2 weeks, in which a participant receives bel-sar injection and laser application (either on a Day 1/Day 2 schedule or same-day schedule) followed by an interval of 12 or 13 days, respectively, prior to the next cycle. Participants in Cohorts 4d, 4e, and 4h will receive 2 treatment cycles (Day 1 bel-sar injection/Day 2 laser application) and participants in Cohort 4j will receive 3 treatment cycles (same-day injection and laser application).

Additionally, inclusion of additional Cohorts 4f, 4g, and 4i will explore neoadjuvant regimen. In the additional cohorts, each treatment cycle constitutes 2 weeks, in which a participant receives bel-sar injection and laser application (either on a Day 1/Day 2 schedule or same-day schedule) followed by an interval of 12 or 13 days, respectively, prior to the next cycle, followed by about 2 weeks of no treatment, followed by TURBT. Participants in Cohorts 4f, 4g, and 4i will receive 2 treatment cycles (Day 1 bel-sar injection/Day 2 laser application), followed by about 2 weeks of no treatment, followed by TURBT.

### Participant Population and Longitudinal Response Assessments

Cohorts 4d, 4e, 4g, and 4h will enroll participants with recurrent low-grade (LG) intermediate-risk (IR) NMIBC. Cohorts 4f and 4i will enroll participants with high-risk (HR) NMIBC. After evaluation of bel-sar in NMIBC cohorts, an MIBC cohort dosed with an optimal treatment regimen as determined from the NMIBC cohorts, will be enrolled.

Standard TURBT will no longer be required as a part of this trial for Cohorts 4d, 4e, and 4h, based on available data from the trial showing preliminary drug activity. Instead, clinical CR will be assessed as an efficacy endpoint, at the 3-month mark based upon visualization on cystoscopy and urine cytology. TURBT may be performed at any time based upon the Investigator's clinical assessment and should be considered for any participant not achieving a CR. Durability of response will be subsequently assessed at the 6-, 9-, and 12-month timepoints.

Standard TURBT will be required as part of this trial for Cohorts 4g, 4f, and 4i, where tumors will be treated with bel-sar first ahead of TURBT to prevent recurrence and progression by treating the tumor first and generating CMI. Participants will undergo TURBT prior to the 3-month mark. Durability of response will be subsequently assessed at the 3-, 6-, 9-, and 12-month timepoints. Cystectomy will still be required for the MIBC cohort. Response will be assessed at the time of cystectomy utilizing histopathological assessment.

Option for Re-Treatment Upon Recurrence after 3-Month CR

For participants enrolled in Cohorts 4d, 4e, and 4h who achieve an initial 3-month CR, it is possible a recurrence may be identified during the follow-up period (up to the 12-month assessment). In this instance, Investigators may administer an additional single cycle of bel-sar treatment at the dose and regimen of the participant's assigned cohort; after which, participants will be followed with cystoscopy and urine cytology every 3 months. Total time followed for this trial will not exceed 15 months post first bel-sar treatment cycle. If the participant does not achieve a CR after this single re-treatment, the Investigator will initiate SoC including but not limited to TURBT.

### Updated Injection Strategy: Injecting Multiple Lesions to Achieve a Whole-Bladder Response

The updated trial design reflects a change in approach with respect to the number of lesions being injected in Cohorts 4d-j and the MIBC Cohort. Instead of injecting a single lesion in the bladder (as previously done in Cohorts 4a-c), up to 3 tumors, starting with the largest, will be injected at each treatment at an assigned cohort-specific per-lesion dose, with the goal of achieving a whole-bladder response.

### Overview of Continued Dose Escalation Design

Cohorts 4d-i will enroll participants receiving sequentially escalating per-lesion dose administrations of 200, 400, and 800 μg of bel-sar (Cohorts 4d, 4e-g, and 4h-i, respectively) to determine the total bladder MTD. Cohorts 4d-h (bel-sar injection Day 1, laser application Day 2) and 4j (same-day bel-sar injection and laser application) will determine both the optimal laser schedule and whether 3 cycles (Cohort 4j) of bel-sar treatment may be more efficacious compared to 2 (Cohort 4d-h).

Safety monitoring will be conducted throughout the trial. Each treatment cycle will be initiated upon the Investigator's clinical judgement after review of safety data from the prior treatment cycle. Safety Review Boards (SRBs) for each cohort will be conducted to review the safety data before proceeding to the next dose level.

After an MTD and an optimal treatment regimen is determined in the NMIBC cohorts, enrollment into the MIBC cohort using a treatment regimen recommended by the SRB may be initiated. A final SRB will be conducted to provide a safety review of all participants enrolled.

Cohort 1b—Injection Only with TURBT (Approximately 5 NMIBC Participants):

Enrollment into this Cohort is complete. This cohort was designed to evaluate the feasibility, safety, and distribution of bel-sar alone (50 μg IM and 50 μg IT) followed by TURBT approximately 24-48 hours post injection in participants with NMIBC.

Cohort 4a-c—Injection(s) and Laser Treatment with TURBT (11 Participants with NMIBC):

Enrollment into these cohorts is complete. These cohorts were designed to evaluate the feasibility, safety, focal bel-sar distribution, focal tumor necrosis, and preliminary drug activity of single doses of bel-sar injection(s) (IT with or without IM injection) followed by laser application.

### Single-Dose Cohorts

Cohort 4a—4 participants were enrolled and received bel-sar injections of 50 μg IT and 50 μg IM on Day 1, laser application on Day 2, and TURBT on Day 9 (+1 day).
Cohort 4b—3 participants were enrolled and received bel-sar IT injection(s) of 100 μg on Day 1, laser application on Day 2, and TURBT on Day 14 (+7 days).
Cohort 4c—4 participants were enrolled and received bel-sar IT injection(s) of 200 μg on Day 1, laser application on Day 2, and TURBT on Day 14 (+7 days).

### 2-Cycle Treatment Cohorts (Approximately 24-Hour Interval Between Injection and Laser Application)

Cohort 4d, 4e, and 4h—Injection(s) with Laser Treatment and Option for TURBT after 3-Month Response Assessment (Up to 24 Participants with Recurrent LG IR NMIBC):

These cohorts will evaluate the feasibility, safety, focal tumor necrosis, and preliminary efficacy of multiple treatment cycles of bel-sar IT injection(s) followed by laser application. Each treatment cycle (bel-sar injections and laser applications) will be 2 weeks long. Biopsies will not be required to assess response, but the treating Investigator may elect to perform a for-cause biopsy or TURBT based upon their visual findings at any point during the trial.

Cohorts 4g, 4f, and 4i—Injection(s) with Laser Treatment and TURBT Before 3-Month Response Assessment (Up to 26 Participants with HR NMIBC):

These cohorts will evaluate the feasibility, safety, focal tumor necrosis, and preliminary efficacy of multiple treatment cycles of bel-sar IT injection(s) followed by laser application and TURBT.

Each treatment cycle (bel-sar injections and laser applications) will be 2 weeks long. All participants will undergo TURBT following last treatment cycle prior to 3-month response assessment.
Cohort 4d—3 (up to 6) participants will receive 2 cycles of treatment with bel-sar IT injection(s) of 200 μg per lesion (up to 3 lesions), per treatment. Participants will undergo bel-sar injection(s) on Days 1 and 15 (+2 days) with laser application the day after each bel-sar injection.
Cohort 4e—3 (up to 6) participants will receive 2 cycles of treatment with bel-sar IT injection(s) of 400 μg per lesion (up to 3 lesions), per treatment. Participants will undergo bel-sar injection(s) on Days 1 and 15 (+2 days), with laser application the day after each bel-sar injection.
Cohort 4f—5 participants (with option for 5 additional participants) with high risk NMIBC will receive 2 cycles of treatment with bel-sar IT injection(s) of 800 μg per lesion (up to 3 lesions), per treatment. Participants will undergo bel-sar injection(s) on Days 1 and 15 (+2 days), with laser application the day after each bel-sar injection. Participants will undergo TURBT before 3-month follow up.
Cohort 4g—5 participants with intermediate risk NMIBC will receive 2 cycles of treatment with bel-sar IT injection(s) of 400 μg per lesion (up to 3 lesions), per treatment. Participants will undergo bel-sar injection(s) on Days 1 and 15 (+2 days), with laser application the day after each bel-sar injection. Participants will undergo TURBT before 3-month follow up.
Cohort 4h—6 participants will receive 2 cycles of treatment with bel-sar IT injection(s) of 800 μg per lesion (up to 3 lesions), per treatment. Participants will undergo bel-sar injection(s) on Days 1 and 15 (+2 days), with laser application the day after each bel-sar injection.
Cohort 4i—6 participants with high risk NMIBC will receive 2 cycles of treatment with bel-sar IT injection(s) of 800 μg per lesion (up to 3 lesions), per treatment. Participants will undergo bel-sar injection(s) on Days 1 and 15 (+2 days), with laser application the day after each bel-sar injection. Participants will undergo TURBT before 3-month follow up.

### 3-Cycle Treatment Cohort (Same-Day Injection and Laser Application)

Cohort 4j—6 participants will receive 3 cycles of treatment with bel-sar IT injection(s) of 800 μg per lesion (up to 3 lesions), per treatment. Participants will undergo bel-sar injection(s) on Days 1, 15 (+2 days), and 29 (+7 days), with laser application at least 3 hours (but no more than 10 hours) after each bel-sar injection.

Cohorts 4e, 4f and 4g are designed to enroll in parallel, at the same per-lesion dose when feasible, to enable a comparative assessment of safety, tolerability, and efficacy between a 2-cycle treatment schedule (Cohort 4e, 4g, and 4f) compared to a 3-cycle same-day injection/laser application schedule (Cohort 4j).

MIBC Cohort—Injection(s) and Laser Treatment with Cystectomy (3 Participants with MIBC):

This cohort will evaluate the feasibility, safety, distribution, focal tumor necrosis, and preliminary drug activity of bel-sar administered at a treatment regimen recommended by the SRB after evaluation of the NMIBC cohort data.

Approximately 3 participants with MIBC will be enrolled after SRB approval following the Cohort 4j SRB review.

If the treating Investigator elects for TURBT or bladder biopsies, at any timepoint during cystoscopic assessment of response, all pathologic specimens will be submitted to the central pathology laboratory. Bel-sar IM injections will be performed in a similar manner and with a similar injection device as other drugs routinely injected into the bladder wall (e.g., Botox injections to treat overactive bladder). IT injections of bel-sar will be injected directly into the intact tumor at the tumor base. Participants may receive SoC therapy including, but not limited to, adjunctive care with intravesical chemotherapy, Bacillus Calmette-Guerin (BCG) therapy, or systemic adjunctive therapy, per Investigator judgment following SoC TURBT or after the 3-month cystoscopic assessment (based upon the participants assigned cohort).

### Design by Cohort:

Enrollment in Cohorts 5a-b will be closed with the implementation this Amendment (Protocol version 6). At the time of this amendment, no participants were enrolled in these cohorts.

Cohort 1b—Injection Phase (IT and IM Injections) with TURBT

Enrollment included 5 participants who meet the selection criteria. Prior to enrollment, participants had a biopsy to diagnose bladder cancer and had NMIBC per the Investigator's clinical judgement. If eligible and needing TURBT per SoC, participants were enrolled into the Injection Phase of the trial and received bel-sar injection alone (without laser application).

Biopsy of the selected tumor was performed as part of the injection procedure on Day 1/Visit 1 and in a location away from where the injection was performed to not impact the distribution at the injection site(s). When additional tumors were present and accessible, a biopsy of the un-injected tumor was taken at the same time (when feasible). The pre-treatment biopsy of the selected tumor (and any other un-injected tumor biopsy if available) were prepared according to the Surgery and Pathology Manual. Biopsy/biopsies were assessed for the presence and composition of immune cells in the tumor prior to treatment as described in the Pathology Specimen Handling and Analysis Plan.

Injections of bel-sar occurred on Day 1/Visit 1. Participants received IT and IM bel-sar injection of (a) 50 μg in 250 μL IT at the tumor edge (in lamina propria) and (b) 50 μg in 250 μL IM at the base of the tumor (above the area of lamina propria injection) with a flexible cystoscope. Injection locations were marked using a consistent technique as outlined in the Surgery and Pathology Manual.

TURBT was performed within 24-48 hours after the bel-sar injections. Injection locations were marked using a consistent technique as outlined in the Surgery and Pathology Manual. Care was taken to minimize as much as possible cauterization damage to the tissue during the TURBT to preserve the architecture of the tumor and surrounding bladder tissue for assessment of bel-sar distribution around the injection site and under the tumor (although some damage was expected). If a biopsy was obtained from an additional distant tumor prior to injection, a second biopsy was obtained from the same additional tumor to assess immune response.

The tumor and resected bladder tissue sample were prepared as instructed in the Surgery and Pathology Manual for histological analysis to assess for focal tumor necrosis and IHC staining to assess distribution of bel-sar in the bladder wall and tumor. The tumor was also assessed histologically for evidence of an immune reaction. A comparison of the pretreatment biopsy from the selected tumor and resected samples was made.

Follow-up included safety visits for Cohort 1b.

Safety measures included observation of the first participant treated with bel-sar for a minimum of 1 day after injection before the next participant was treated to allow a safety assessment of the injection. The SRB approved the initiation of subsequent Cohorts based on the safety and tolerability of bel-sar and the feasibility of the IT and IM injection procedures. A further safety review was completed after 3 participants received treatment and were followed for 28 days.

Cohort 4a, 4b, and 4c—“Single-Cycle Treatment” Cohorts: Injection and Laser Phase with TURBT (IT and IM Injections)

Enrollment in this expanded cohort was initiated after feasibility and safety were assessed and confirmed (28 days after the 3^{rd} participant in Cohort 1b was treated). Ten participants who meet the eligibility criteria (i.e., participants with NMIBC and planned TURBT per SoC) were enrolled.

Pre-Injection Biopsy of the target tumor (tumor selected for injection) was performed as part of the procedure on Day 1/Visit 1 in a location away from where the injection was to be performed as to not impact distribution at the injection site(s). However, if the participant underwent a biopsy (pathologic confirmation of NMIBC diagnosis) in the 6 weeks immediately prior to Day 1/Visit 1, then the pre-injection biopsy can be omitted.

When an additional tumor was present and accessible (non-target tumor), a biopsy of the non-target tumor was taken at the same time, when feasible. The biopsy specimen(s) were prepared as instructed in the Surgery and Pathology Manual.

Injections of bel-sar occurred on Day 1/Visit 1.
Cohort 4a—4 participants received IT and IM bel-sar injections of (a) 50 μg in 250 μL IM at the tumor edge (in the lamina propria) and (b) 50 μg in 250 μL IT at the base of the tumor (above the area of lamina propria injection) with a flexible or rigid cystoscope.

Injection locations were marked using a consistent technique as outlined in the Surgery and Pathology Manual.
Cohort 4b—3 participants received bel-sar IT injection(s) of 100 μg at the base of the tumor. Participants with a target tumor <2 cm received a single bel-sar IT injection of 100 μg at the base of the tumor, while participants with a target tumor ≥2 cm received 2 separate injections of 50 μg each at opposite sides of the tumor base (when feasible).

For participants with a target tumor ≥2 cm, the total dose (either 100 μg or 200 μg) was split across 2 separate IT injections as shown below (IP Administration By Tumor Size-Cohorts 4b and 4c). Injection locations were marked using a consistent technique as outlined in the Surgery and Pathology Manual.
Cohort 4c—3 participants received bel-sar IT injection(s) of 200 μg at the base of the tumor. Participants with a target tumor <2 cm received a single bel-sar IT injection at the base of the tumor, while participants with a target tumor ≥2 cm received 2 separate injections at opposite sides of the tumor base (when technically feasible). For those participants with a target tumor ≥2 cm, the total dose (200 μg) was split across 2 separate IT injections as shown below-IP Administration By Tumor Size (Cohorts 4b and 4c)

Injection locations should be marked using a consistent technique.

IP Administration By Tumor Size (Cohorts 4b and 4c)
Participants receiving 100 OR 200 μg of bel-sar IT

| bel-sar | | | |
|---|---|---|---|
| Total Dose | Tumor Size | | bel-sar Injection(s) |
| 100 μg | <2 | cm | 100 μg IT (at the base of tumor) |
| 100 μg | ≥2 | cm | 50 μg IT (at the base of the tumor) |
| | | | 50 μg IT (at the base of the tumor) |
| | | | Inject at opposite sides of the tumor |
| | | | base (if technically feasible). |
| 200 μg | <2 | cm | 200 μg IT (at the base of tumor) |
| 200 μg | ≥2 | cm | 100 μg IT (at the base of the tumor) |
| | | | 100 μg IT (at the base of the tumor) |
| | | | Inject at opposite sides of the tumor |
| | | | base (if technically feasible). |

Laser application occurred on Day 2/Visit 2 via flexible or rigid cystoscopy. Planning allowed for the laser application to occur approximately 24 hours after bel-sar injection.

### TURBT

Cohort 4a—(50 μg IT and 50 μg IM)—TURBT occurred on Day 9 (±1 day). Care was taken to minimize cauterization damage to the tissue during the TURBT to preserve the architecture of the target tumor and surrounding bladder tissue for assessment of bel-sar distribution around the injection site and under the target tumor. If a biopsy was obtained from a non-target tumor prior to injection, a second biopsy was obtained from the same non-target tumor to assess immune response.
Cohorts 4b and 4c—(100 μg and 200 μg) TURBT occurred on Day 14 (+7 days)/Visit 3. Care was taken to minimize cauterization damage to the tissue during the TURBT to preserve the architecture of the target tumor and surrounding bladder tissue for assessment of bel-sar distribution around the injection site and under the target tumor. If a biopsy was obtained from a non-target tumor prior to injection, a second biopsy was obtained from the same non-target tumor to assess immune response.

The tumor and resected bladder tissue sample for all dose schedules were prepared as instructed in the Surgery and Pathology Manual. Participants received SoC post-operatively.

Follow-up includes safety visits for Cohorts 4a, 4b, and 4c.

PK and anti-drug antibody (ADA) samples are being collected to confirm no detection of bel-sar.

Safety measures included SRB assessment (SRB C4a) of local and systemic safety (based on AEs/SAEs and clinical assessments) 28 days after the 3rd participant in Cohort 4a was treated with bel-sar injections of 50 μg IT+50 μg IM with laser application and included an evaluation of potential DLTs. After SRB C4a. 3 additional participants were enrolled in Cohort 4b to receive bel-sar 100 μg IT injection and safety review (SRB C4b) occurred after 3 participants received bel-sar 100 μg IT injection with laser application and were followed for 28 days. After SRB C4b, 4 additional participants were enrolled in Cohort 4c and received bel-sar IT injection of 200 μg with laser application.

Cohorts 4d, 4e, 4g and 4h (Recurrent Low-Grade, Intermediate Risk NMIBC) Cohorts 4f, and 4i (High Risk NMIBC)—“2-Cycle Treatment” Cohorts (4d-i) and “3-Cycle Treatment” Cohort (4j):

Enrollment in Cohort 4d will be initiated after assessment and confirmation of the feasibility and safety from a single 200 μg dose of bel-sar (28 days after at least the 3^{rd} participant in Cohort 4c is treated [SRB C4c]). Enrollment in Cohort 4e will be initiated after assessment and confirmation of the feasibility and safety of Cohort 4d treatment is confirmed by SRB C4d. Enrollment in Cohorts 4f and 4g will occur in parallel and will be initiated after confirmation of feasibility and safety of Cohort 4e treatment by SRB C4e. Approximately 18 (up to 24) participants who meet the eligibility criteria (i.e., participants with recurrent LG IR NMIBC) will be enrolled across cohorts 4d-4g. While there is no restriction on the number of lesions to be enrolled in the study, a maximum of 3 lesions may be injected per treatment (these need not be the same tumors treated in a previous cycle).

Pre-Injection Biopsy: at least 1 target tumor (tumor selected for injection) should be performed as part of the procedure on Day 1/Visit 1 in a location away from where the injection is to be performed to not impact distribution at the injection site(s). If there is more than 1 target tumor, biopsy should also be performed as part of the procedure. However, if the participant underwent a biopsy (pathologic confirmation of recurrent LG IR NMIBC diagnosis) within the 8 weeks immediately prior to Day 1/Visit 1, then the pre-injection biopsy can be omitted. Tissue samples from this previous biopsy must be submitted to the Central Pathology Lab.

If an additional tumor is present and accessible (non-target tumor), a biopsy of the non-target tumor should be taken at the same time, if feasible. The biopsy specimen(s) will be prepared as instructed in the Surgery and Pathology Manual.

Bel-sar Injections and “Total Bladder Dose” per treatment: At each treatment cycle, a maximum of 3 lesions will be injected at the cohort-assigned per-lesion dose, per tumor, starting with the largest tumor. Therefore, the maximum total bladder dose per cycle is equal to 3 times the dose per lesion. For example, in Cohort 4d, in a participant with 3 tumors, the total bladder dose per treatment will be 3×200 μg=600 μg. At the time of second cycle of treatment (or third cycle for Cohort 4j), up to 3 tumors may be treated again based on Investigator discretion, starting with the largest tumor. These need not be the same tumors treated in the first cycle. In participants with less than or equal to 3 lesions, all lesions should be treated: in participants with more than 3 lesions a maximum of 3 lesions may be treated.

The number and locations of tumors selected for injection, as well as the number and locations of injections in each tumor should be documented in source at each treatment as outlined in the Surgery and Pathology Manual. Should there be greater than 3 tumors identified at time of treatment, the tumors that were not injected with bel-sar will be considered “non-target”.

Further details on how the intratumoral injections will be performed (such as overall objective, number and volume of injections) are provided in the Pharmacy Manual. Injection locations should be marked using a consistent technique as outlined in the Surgery and Pathology Manual.

Treatment Cycle: a treatment cycle is defined as a 2-week period in which participants will receive bel-sar injection and laser application at the beginning of the 2-week period.

### Cohort 4d (200 μg Bel-Sar Per Lesion, 600 μg Max Dose Per Cycle, 2 Cycles)

6 participants will receive IT bel-sar injections of 200 μg at the base of the tumor (up to 3 lesions) with a flexible or rigid cystoscope on Day 1 and Day 15 (+2 days). Laser application will occur on the day after each bel-sar injection (approximately 24 hours after the injection).

### Cohort 4e (400 μg Bel-Sar Per Lesion, 1200 μg Max Dose Per Cycle, 2 Cycles)

5 participants will receive IT bel-sar injections of 400 μg at the base of the tumor (up to 3 lesions) with a flexible or rigid cystoscope on Day 1 and Day 15 (+2 days). Laser application will occur on the day after each bel-sar injection (approximately 24 hours after the injection).

### Cohort 4g (400 μg Bel-Sar Per Lesion, 1200 μg Max Dose Per Cycle, 2 Cycles)

5 participants will receive IT bel-sar injections of 400 μg at the base of the tumor (up to 3 lesions) with a flexible or rigid cystoscope on Day 1 and Day 15 (+2 days). Laser application will occur on the day after each bel-sar injection (approximately 24 hours after the injection). Participants will undergo TURBT 2 weeks after last laser application before the 3-month response assessment.

### Cohort 4f (400 μg Bel-Sar Per Lesion, 1200 μg Max Dose Per Cycle, 2 Cycles)

5 participants (with option for 5 additional participants) will receive IT bel-sar injections of 400 μg at the base of the tumor (up to 3 lesions) with a flexible or rigid cystoscope on Day 1 and Day 15 (+2 days). Laser application will occur on the day after each bel-sar injection (approximately 24 hours after the injection). Participants will undergo TURBT 2 weeks after last laser application before the 3-month response assessment.

### Cohort 4h (800 μg Bel-Sar Per Lesion, 2400 μg Max Dose Per Cycle, 2 Cycles)

6 participants will receive IT bel-sar injections of 800 μg at the base of the tumor (up to 3 lesions) with a flexible or rigid cystoscope on Day 1 and Day 15 (+2 days). Laser application will occur on the day after each bel-sar injection (approximately 24 hours after the injection).

### Cohort 4i (800 μg Bel-Sar Per Lesion, 2400 μg Max Dose Per Cycle, 2 Cycles)

6 participants will receive IT bel-sar injections of 800 μg at the base of the tumor (up to 3 lesions) with a flexible or rigid cystoscope on Day 1 and Day 15 (+2 days). Laser application will occur on the day after each bel-sar injection (approximately 24 hours after the injection). Participants will undergo TURBT 2 weeks after last laser application before the 3-month response assessment.

### Cohort 4j (800 μg Bel-Sar Per Lesion, 2400 μg Max Dose Per Cycle, 3 Cycles)

6 participants will receive IT bel-sar injections of 800 μg at the base of the tumor (up to 3 lesions) with a flexible or rigid cystoscope on Day 1. Day 15 (+2 days), and Day 29 (+7 days). Laser application will occur on the same day as each bel-sar injection (at least 3 hours post injection, but no longer than 10 hours post injection).

Participants in Cohorts 4e, 4g, and 4f will be enrolled in parallel and at the same per lesion dose, where feasible.

### Response Assessments

### Cohorts 4d, 4e, and 4h:

Visit 3: in-clinic response assessments include white light cystoscopy, individual tumor photographs (video optional), and documentation of the diameter of each visible tumor with optional for-cause biopsies and urine cytology.

Visit 5 (3-month follow-up [FU]): in-clinic response assessments include white light cystoscopy, individual tumor photographs (video optional), documentation of the diameter of each visible tumor, and urine cytology. Should there be visual evidence of disease or indeterminate visual findings, a biopsy of each tumor is required. After the completion of all treatment cycles, cystoscopies may be performed as clinically indicated upon Investigator discretion before the 3-mth FU response assessment.

Visit 6, 7, and 8 (6-, 9-, and 12-month FU): in-clinic response assessments include white light cystoscopy, individual tumor photographs (video optional), documentation of the diameter of each visible tumor, and urine cytology. Should there be visual evidence of disease or indeterminate visual findings, a biopsy of each tumor is required. Cystoscopies may be performed as clinically indicated upon Investigator discretion in between these time points.

Additional urine biomarkers will be collected and may be evaluated during the trial to assess prognostic and predictive markers of treatment response and outcomes and to assess changes in molecular tumor profiles.

### Cohorts 4g, 4f, and 4i:

Visit 3: in-clinic response assessments include white light cystoscopy, individual tumor photographs (video optional), and documentation of the diameter of each visible tumor with optional for-cause biopsies and urine cytology.

Visit 5: TURBT will be performed prior to the 3-month follow-up (visit 4). Care should be taken to minimize possible cauterization damage to the tissue during the TURBT to preserve the architecture of the tumor and surrounding bladder tissue for assessment of bel-sar distribution around the injection site and under the tumor. If a biopsy was obtained from an additional distant tumor prior to injection, a second biopsy should be obtained from the same additional tumor to assess immune response. If a biopsy was obtained from a non-target tumor prior to injection, a biopsy TUR specimen should also be obtained from the same non-target tumor to assess immune response.

The tumor and resected bladder tissue sample for all dose schedules will be prepared and submitted to the Central Pathology Laboratory as instructed in the Surgery and Pathology Manual. Participants should receive SoC post-operatively.

Visit 6 (3-month follow-up [FU]): in-clinic response assessments include white light cystoscopy, individual tumor photographs (video optional), documentation of the diameter of each visible tumor, and urine cytology. Should there be visual evidence of disease or indeterminate visual findings, a biopsy of each tumor is required. After the completion of all treatment cycles, cystoscopies may be performed as clinically indicated upon Investigator discretion before the 3-mth FU response assessment.

Visit 7, 8, and 9 (6-, 9-, and 12-month FU): in-clinic response assessments include white light cystoscopy, individual tumor photographs (video optional), documentation of the diameter of each visible tumor, and urine cytology. Should there be visual evidence of disease or indeterminate visual findings, a biopsy of each tumor is required. Cystoscopies may be performed as clinically indicated upon Investigator discretion in between these time points.

Additional urine biomarkers will be collected and may be evaluated during the trial to assess prognostic and predictive markers of treatment response and outcomes and to assess changes in molecular tumor profiles.

### Cohort 4j:

Visit 2 and 3: in-clinic response assessments include white light cystoscopy, individual tumor photographs (video optional), and documentation of the diameter of each visible tumor, with optional for-cause biopsies and urine cytology.

Visit 4 (3-month FU): in-clinic response assessments include white light cystoscopy, individual tumor photographs (video optional), documentation of the diameter of each visible tumor and urine cytology. In addition, should there be visual evidence of disease or indeterminate visual findings, a for-cause biopsy of each tumor is required. After the completion of all treatment cycles, cystoscopies may be performed as clinically indicated at the discretion of the treating Investigator before the 3-month FU response assessment

Visit 5, 6, and 7 (6-, 9-, and 12-month FU): in-clinic response assessments include white light cystoscopy, individual tumor photographs (video optional), documentation of the diameter of each visible tumor, and urine cytology. Should there be visual evidence of disease or indeterminate visual findings, a biopsy of each tumor is required. Cystoscopies may be performed as clinically indicated upon Investigator discretion in between these time points

Additional urine biomarkers may be evaluated during the trial.

Safety Review: AEs, SAEs (including ADEs and SADEs) and DLTs will be reviewed at an SRB after 3 participants in cohorts 4d and 4e and after at least 3 participants in cohort 4f have completed the DLT period (14 days after last treatment) of that cohort.

Recurrences after Complete Response: For participants enrolled in Cohorts 4d-j who achieve an initial 3-month CR, it is possible that a recurrence may be identified while followed in this trial at the 6-, 9-, or 12-month assessment. Investigators are permitted to offer a single bel-sar treatment cycle in this scenario. The additional cycle dose will match the treatment regimen of the assigned cohort. Participants will then be followed with cystoscopy and urine cytology every 3 months.

TURBT may be performed at any point during the study, including the 3-month response assessment at the discretion of the treating Investigator. Care should be taken to minimize possible cauterization damage to the tissue during the TURBT to preserve the architecture of the tumor and surrounding bladder tissue for assessment of bel-sar distribution around the injection site and under the tumor. If a biopsy was obtained from an additional distant tumor prior to injection, a second biopsy should be obtained from the same additional tumor to assess immune response. If a biopsy was obtained from a non-target tumor prior to injection, a biopsy TUR specimen should also be obtained from the same non-target tumor to assess immune response.

The tumor and resected bladder tissue sample for all dose schedules will be prepared and submitted to the Central Pathology Laboratory as instructed in the Surgery and Pathology Manual. Participants should receive SoC post-operatively.

Follow-up includes 3, 6, 9, and 12-month visits for Cohorts 4d-j. Between the end of the treatment cycles to the 3-month FU timepoint, clinical assessment of response is not mandated, but can be performed as clinically indicated.

PK, ADA and Biomarker samples will be collected for Cohorts 4d-g.

Safety measures include conducting an SRB after each cohort, where local and systemic safety will be assessed. For Cohorts 4d and e, SRBs will be conducted after 3 participants in each cohort have completed the DLT period, before enrollment into the next cohort can be initiated. For Cohorts 4f and 4g, SRB will be conducted after at least 3 participants have completed the DLT period in each cohort.

MIBC Cohort (MIBC)—Treatment Regimen and Injection-Laser Schedule Determined from NMIBC Cohorts

Enrollment in the MIBC cohort may be initiated after the Cohort 4h, 4i, or 4j SRB, where the totality of safety data from all NMIBC participants enrolled in the dose escalation/laser application optimization Cohorts (Cohorts 4a-j) will be reviewed. The SRB will determine the MTD in NMIBC participants. In addition, the SRB may recommend an alternative dose from the MTD that could be considered safe and tolerable for the treatment of MIBC participants with potentially higher tumor loads. In addition, based on depth of tumor infiltration, the optimal injection and laser schedule in MIBC may differ from treatment in NMIBC. Approximately 3 participants may be enrolled into the MIBC cohort.

Pre-Injection Biopsy, Injections, Laser Application Will Follow that of the Optimized Treatment Regimen and Schedule Determined from NMIBC Cohorts.

Response Assessments: At the time of cystectomy, histopathological evaluation of the cystectomy specimen plus pelvic lymph nodes, if indicated, will be conducted.

Cystectomy will occur per SoC after a minimum of 14 days post last treatment. The resection specimen will be prepared as instructed in the Surgery and Pathology Manual. Participants should receive SoC post-operatively.

Follow-up will include a safety visit at the time of cystectomy.

PK, ADA and Biomarker samples will be collected.

Safety review: An SRB may be conducted after all participants in the MIBC cohort have completed scheduled bel-sar treatments and have been followed for at least 14 days after the last laser treatment. In the absence of a dedicated SRB for the MIBC cohort, upon conclusion of the study, the SRB will conduct a final review, including the safety data from the MIBC cohort, to assess overall safety data for all participants.

### Participation Duration:

For Cohorts 1b, 4a-c: Participants will be followed for at least 56 days for safety. Total time for each participant (i.e., from the initiation of screening until the End of Trial [EoT] visit) including all safety follow-up is approximately 84 days.
For Cohorts 4d-g: Total time for each participant (i.e., from initiation of screening until the EoT visit) including all safety follow-up is approximately 393 days. Participants who have re-treatment at the 12-mth FU timepoint, will have an additional follow-up of 90 days.
For the MIBC Cohort: Participants will be followed for at least to 56 days for safety, depending on which treatment regimen is recommended for the MIBC cohort. Total time for each participant (i.e., from the initiation of screening until the EoT visit) including all safety follow-up is approximately 84 days.

### Trial Methods:

Participants will be screened and enrolled based upon eligibility criteria and after providing written informed consent between Day −28 and Day −1. Screening procedures include medical history, surgical history, urologic assessments, risk assessments, vital signs, and safety laboratory analyses. To be eligible for enrollment, participants must have a diagnosis of urothelial carcinoma.

Once all eligibility criteria are confirmed, participants will be assigned to a cohort based on available openings. Participants will receive treatment and be followed for up to 3 months post initiation of bel-sar treatment.
Treatment-emergent and treatment-related AEs.
Concomitant medications.
Medical history/Surgical history.
Risk assessment and NMIBC/MIBC status.
Hematology and serum chemistry (complete blood count with differential and Comprehensive Metabolic Panel).
Blood sample collection for PK analysis.
Blood sample collection for ADA analysis.
Blood sample collection for Biomarker analysis.
Urinalysis
Electrocardiogram.
Physical Examination (PE).
Eastern Cooperative Oncology Group Performance Status Scale.
Vital signs (blood pressure, temperature, pulse, and respiration) including height and weight (body mass index will be calculated).
Flexible or rigid cystoscopy with bel-sar injection(s) (all cohorts).
Flexible or rigid cystoscopy with laser application (Cohorts 4a-g and MIBC cohort).
Cystoscopy to assess for clinical response
Biopsies:
   Pre-injection biopsy of at least 1 target tumor at a location away from the site of injection, unless there has been pathologic confirmation of recurrent LG IR NMIBC diagnosis in the 8 weeks immediately prior to Day 1/Visit 1 and tissue from these samples is available to be submitted to the Central Pathology Laboratory.
   Biopsy of a non-target tumor, if present and technically feasible.
   For-cause biopsies to confirm presence or absence of urothelial carcinoma
Immune biomarker assessment of biopsy tissue
TURBT (Cohorts 1b and 4a-c, 4g, 4f, and 4i); at the discretion of the treating investigator for Cohorts 4d, 4e, and 4h.
Cystectomy (MIBC cohort only).
Digital photography and optional video of the injection procedure.
Urine cytology.
Urine biomarkers.
Unstructured Participant Feedback
All treatment procedures must be performed by Investigators fully trained in those procedures or delegated to appropriate personnel fully trained on the procedure(s).

### Inclusion Criteria:

Participants must:
1. Be at least 18 years of age.
2. Have been informed about the investigative nature and requirements of the trial, voluntarily agreed to participate in the trial and have documented their consent by signing and dating the Informed Consent Form before participating in any trial-related activities.
3. Meet the following histopathologic requirements for urothelial carcinoma:
   For Cohorts 1b, 4a-c, histopathological diagnosis of NMIBC (any grade) is required. For participants with first diagnosis of NMIBC, confirmation of urothelial carcinoma by recent biopsy (≤6 months of Screening Visit) is required with clinical appearance of NMIBC based on Investigator judgement. Participants with recurrent NMIBC must have a current lesion that clinically appears to be NMIBC (per the Investigator's judgement based on cystoscopy) with histopathologic confirmation based on TURBT or biopsy within the last 24 months).
   For Cohorts 4d, 4e, 4g, diagnosis of recurrent low-grade intermediate risk NMIBC (according to AUA risk classification guidelines) is required, specifically:
      Single or multiple low grade Ta tumor(s) that have recurred in past year OR
      Solitary low grade Ta tumor >3 cm with recurrence
   For Cohorts 4f, 4h, and 4i, diagnosis of high risk NMIBC (according to AUA risk classification guidelines) is required.
   In addition, participants:
      Must have a current lesion that clinically appears to be NMIBC (per the Investigator's judgement based on cystoscopy) with histopathologic confirmation based on TURBT or biopsy (within the last 24 months).
      Must have bladder cancer tumors (papillary lesions or broader sessile lesions) identified at Screening available for bel-sar injection. While there is no restriction on number of lesions, a maximum of 3 lesions may be injected per treatment (these need not be the same tumors treated in a previous cycle).
      Must not have high grade urothelial carcinoma on screening cytology. For MIBC cohort, MIBC must be demonstrated pathologically. For participants with first diagnosis of MIBC, confirmation of urothelial carcinoma with muscle-invasion by recent resection biopsy (≤6 months of Screening Visit) is required with clinical appearance of MIBC based on Investigator judgement. For participants with prior diagnosis of MIBC, participants must have residual MIBC of ≥1 cm per investigator's clinical assessment.
4. Have no evidence of current or prior metastatic urothelial carcinoma.
5. Have a diagnosis of NMIBC or MIBC:
   a. Cohorts 1b and Cohort 4a-c, 4g, 4f, and 4i require TURBT per Investigator's SoC and judgement.
   b. MIBC cohort requires cystectomy per Investigator's SoC and judgment (i.e., high risk NMIBC with BCG failure, or carcinoma in situ, or participants with MIBC who are not eligible to receive neoadjuvant chemotherapy per Investigator's SoC).
   c. For Cohorts 4a-c, one of the tumors (papillary or carcinoma in situ lesions) must be ≥1.0 cm in greatest dimension and must be accessible for bel-sar injection per Investigator judgement.
   d. For Cohorts 4d, 4e, 4g, and 4h, participants with recurrent low grade intermediate risk NMIBC and tumors of any size are eligible (no requirements on lesion size)
   e. For Cohorts 4f and 4i, participants with high-risk NMIBC and tumors of any size are eligible (no requirements on lesion size)
6. If female and capable of becoming pregnant, agree to have pregnancy testing performed at screening (must be negative) and if required within 24 hours of bel-sar injection (must be negative); must agree to use highly effective method of contraception from the date of the screening pregnancy test; and must agree to continue using such precautions for 90 days after the final dose of bel-sar. Women considered capable of becoming pregnant include all females who have experienced menarche and have not experienced menopause (as defined by amenorrhea for greater than 12 consecutive months) or have not undergone successful surgical sterilization (hysterectomy, bilateral tubal ligation, or bilateral oophorectomy).
7. If male, must be of non-reproductive potential (i.e., have azoospermia either from a vasectomy or from an underlying medical condition) or must use a highly effective method of contraception defined as one that results in a low failure rate (i.e., less than 1% per year) when used consistently and correctly. Males with sexual partners of childbearing potential must agree to use a highly effective form of barrier birth control as outlined in the protocol or abstain from sexual intercourse during the trial and for 90 days after a bel-sar injection. Males should not donate sperm until 90 days after injection of bel-sar.
8. Be able and willing to avoid all prohibited medications for the appropriate washout period and during trial follow-up.
9. Participants may receive continuous anticoagulation or antiplatelet therapy as medically required (temporary discontinuation is permitted as long as bel-sar injections and laser application procedures can be safely administered and following discussion with the trial's Medical Monitor).
10. Adequate bone marrow, renal, and hepatic function as defined by:

Adequate Organ Function

| | |
|---|---|
| System | Laboratory Values |

Hematologic

| | | |
|---|---|---|
| Absolute Neutrophil Count | >1200 | cells/μL |
| Hemoglobin | >9 | g/dL |

| | |
|---|---|
| Platelets | >75,000/μL |

Hepatic

| | |
|---|---|
| ALT and AST | ≤3.0 × ULN |
| Total bilirubin | ≤1.5 × ULN unless the participant |
| | has documented/suspected |
| | Gilbert's, then ≤3 × ULN |
| Albumin | ≥3.0 g/dL (30 g/L) |

Renal

| | | |
|---|---|---|
| Glomerular Filtration Rate | ≥30 | mL/min |

ALT: alanine aminotransferase; AST: aspartate aminotransferase ULN: upper limit of normal.

### Exclusion Criteria:

### Participant:

Must not have evidence of current or prior metastatic urothelial carcinoma.

Must not have any additional malignancy that requires active treatment, unless deemed appropriate after discussion by the Investigator with the trial's Medical Monitor.

Must not have any contraindication to general or spinal anesthesia.

Is unable to undergo flexible or rigid cystoscopy.

Must not be pregnant or lactating.

Must not have used an investigational drug or medical device within 30 days or 5 half-lives (whichever is longer) of Visit 1 or concurrent enrollment in another investigational trial.

Must not have known contraindications or sensitivities to phthalocyanine-based dye, bel-sar, the capsid component, or prior treatment with laser.

Must not have active autoimmune disease, chronic inflammatory condition, or other conditions (like solid organ transplant or bone marrow allograft) requiring concurrent use of any systemic immunosuppressants or steroids.

Must not have a known active immune-deficiency state(s) or infection state-including, but not limited to active HIV, chronic active Hep B or C, myelodysplastic disorders, marrow failures, history of solid organ transplant, or bone marrow allograft.

Must not have received any systemic anticancer therapy not otherwise mentioned above within the last 28 days.

Must not have active bacterial, fungal, or viral infections-all prior infections must have resolved following optimal therapy and participants must be off all systemic anti-infective agents. Must not have a history of coagulopathy resulting in uncontrolled bleeding, e.g., hemophilia, von Willebrand's disease, etc.

### Cardiac Exclusions:

New York Heart Association Class 3 or 4 congestive heart failure.

Uncontrolled hypertension.

Unstable angina pectoris.

Clinically important cardiac arrhythmia (e.g., requiring anti-arrhythmic drugs like sotolol).

Mean QT interval corrected for heart rate (QTc)>470 ms calculated from an ECG using

Fredericia's Correction by manual read.

Must not have any other condition or previous surgery that, in the opinion of the Investigator, would interfere with trial participation, evaluation of the IP, interpretation of trial results, or put the participant at any unnecessary risk.

### Investigational Product, Dosage, and Mode of Administration:

The investigational treatment includes a drug (bel-sar), and a laser (Laser Photoactivation System), supplied by Aura that delivers 689 nm light to activate the drug once it is bound to the tumor cells. Bel-sar drug substance is a modified human papillomavirus-derived, recombinantly manufactured, empty virus-like particle (VLP) conjugated with approximately 200 molecules of sarotalocan (a phthalocyanine-based dye (the “Dye”). The VLP and Dye combination is called a virus-like drug conjugate.

Bel-sar drug product is a sterile, pale blue aqueous solution supplied at a concentration of 0.4 mg per mL or 1.0 mg per mL of buffered isotonic solution (pH 6.5). Bel-sar is supplied in a 2.0 mL, single-use vial; each vial contains a fill volume of 0.3 mL. Bel-sar will be administered via IT injection with or without IM injection using a cystoscopic approach. For all cohorts except 1b, the Laser Photoactivation System consists of a laser console and a frontal light distributor. The laser light (689 nm) is applied to the bladder tumors.

| Cohort | Bel-sar Injection | Laser Applicationb | Surgery |
|---|---|---|---|
| Cohort 1b | 50 μg in 250 μL IT (at | Not applicable | TURBT within 24 |
| 5 NMIBC | the base of the tumor | | hours after bel-sar |
| participants | above the area of | | injection |
| | lamina propria | | |
| | injection) | | |
| | 50 μg in 250 μL IM (at | | |
| | the tumor | | |
| | edge in lamina propria) | | |
| Cohort 4a | 50 μg in 250 μL IT (at | Administered Day 2 | TURBT Day 9 ± 1 D |
| Approximately | the base of the tumor | | |
| 4 NMIBC | above the area of | | |
| Participants | lamina propria | | |
| | injection) | | |
| | 50 μg in 250 μL IM | | |
| | (at the tumor edge in | | |
| | lamina propria) | | |
| Cohort 4b | 100 μg IT (at the base of | Administered Day 2 | TURBT Day 14 ± 7 D |
| Approximately | the tumor)^{a} | | |
| 3 NMIBC | | | |
| Participants | | | |
| Cohort 4c | 200 μg IT (at the base of | Administered Day 2 | TURBT Day 14 ± 7 D |
| Approximately | the tumor)^{a} | | |
| 3 NMIBC | | | |
| Participants | | | |
| Cohort 4d^{b} | Day 1: 200 μg per lesion | Administered Day 2 | Optional TURBT |
| 3 (up to 6) | IT, up to 3 lesions (at the | Administered Day 16 | after 3-mth |
| Recurrent LG | base of the tumor) | (or day after injection) | response |
| IR NMIBC | Day 15 (+2 days): 200 | | assessment |
| Participants | μg per lesion IT, up to 3 | | |
| | lesions (at the base of the | | |
| | tumor) | | |
| | Max dose per bladder per | | |
| | cycle (3 lesions): 600 μg | | |
| Cohort 4eb | Day 1: 400 μg per lesion | Administered Day 2 | Optional TURBT |
| 3 (up to 6) | IT, up to 3 lesions (at the | Administered Day 16 | after 3-mth |
| Recurrent LG | base of the tumor) | (or day after injection) | response |
| IR NMIBC | Day 15 (+2 days): 400 | | assessment |
| Participants | μg per lesion IT, up to 3 | | |
| | lesions (at the base of the | | |
| | tumor) | | |
| | Max dose per bladder per | | |
| | cycle (3 lesions): 1200 μg | | |
| Cohort 4g^{b} | Day 1: 400 μg per lesion | Administered Day 2 | TURBT before 3- |
| 5 Recurrent | IT, up to 3 lesions (at the | Administered Day 16 | month response |
| LG IR | base of the tumor) | (or day after injection) | assessment |
| NMIBC | Day 15 (+2 days): 400 | | |
| Participants | μg per lesion IT, up to 3 | | |
| | lesions (at the base of the | | |
| | tumor) | | |
| | Max dose per bladder per | | |
| | cycle (3 lesions): 1200 μg | | |
| Cohort 4f^{b} | Day 1: 400 μg per lesion | Administered Day 2 | TURBT before 3- |
| 5 [+5] HR | IT, up to 3 lesions (at the | Administered Day 16 | month response |
| NMIBC | base of the tumor) | (or day after injection) | assessment |
| Participants | Day 15 (+2 days): 400 μg | | |
| | per lesion IT, up to 3 | | |
| | lesions (at the base of the | | |
| | tumor) | | |
| | Max dose per bladder per | | |
| | cycle (3 lesions): 1200 μg | | |
| Cohort 4h^{b} | Day 1: 800 μg per lesion | Administered Day 2 | Optional TURBT |
| 6 Recurrent | IT, up to 3 lesions (at the | Administered Day 16 | after 3-mth |
| LG IR | base of the tumor) | (or day after injection) | response |
| NMIBC | Day 15 (+2 days): 800 | | assessment |
| Participants | μg per lesion IT, up to 3 | | |
| | lesions (at the base of the | | |
| | tumor) | | |
| | Max dose per bladder per | | |
| | cycle (3 lesions): 2400 μg | | |
| Cohort 4i^{b} | Day 1: 800 μg per lesion | Administered Day 2 | TURBT before 3- |
| 6 HR NMIBC | IT, up to 3 lesions (at the | Administered Day 16 | month response |
| Participants | base of the tumor) | (or day after injection) | assessment |
| | Day 15 (+2 days): 800 μg | | |
| | per lesion IT, up to 3 | | |
| | lesions (at the base of the | | |
| | tumor) | | |
| | Max dose per bladder per | | |
| | cycle (3 lesions): 2400 μg | | |
| Cohort 4j^{b, c} | Day 1: 800 μg per lesion | Administered Day 1 | Optional TURBT |
| 6 Recurrent | IT, up to 3 lesions (at the | (3-10 hours after | after 3-mth |
| LG IR | base of the tumor) | injection, same-day) | response |
| NMIBC | Day 15 (+2 days): 800 μg | Administered Day 15 | assessment |
| Participants | per lesion IT, up to 3 | (3-10 hours after | |
| | lesions (at the base of the | injection, same-day) | |
| | tumor) | | |
| | Day 29 (+7 days): 800 μg | Administered Day 29 | |
| | per lesion IT, up to 3 | (3-10 hours after | |
| | lesions (at the base of the | injection, same-day) | |
| | tumor) | | |
| | Max dose per bladder per | | |
| | cycle (3 lesions): 2400 μg | | |

| | | |
|---|---|---|
| MIBC | Treatment regimen and injection-laser schedule | Cystectomy 2 wks |
| Approximately | as determined by NMIBC cohorts | (+14 days) post last |
| 3 MIBC | | bel-sar treatment |
| Participants | | |

TURBT: transurethral resection of the bladder tumor; MIBC: muscle invasive bladder cancer; NMIBC non-muscle invasive bladder cancer; IT: intratumoral; IM: intramural.
^{a}Cohorts 4b and 4c: For participants receiving the 100 μg or 200 μg IT dose with a target tumor ≥2 cm, the dose will be split into 2 equal IT injections administered at opposite sides of the base of the tumor (if technically feasible). For participants with a target tumor <2 cm, a single injection will be administered at the specified dose.
^{b}Cohorts 4d-j: Doses are administered on a per-lesion basis based on the assigned dose level, to achieve a “total bladder dose” per treatment. Up to 3 lesions are to be injected at each treatment, starting with the largest tumor where feasible. An appropriate number of injections per lesion may be administered with the goal of ensuring equal distribution of bel-sar around the tumor base and maximizing exposure of tumor base to bel-sar.
^{c}Cohort 4j: Enrolled in parallel with Cohort 4f, where feasible. Cohort 4j per-lesion dose may be 800 μg, an intermediate dose or MTD, based on emerging safety data from preceding dose levels.

### Statistical Methods:

### General Considerations:

Continuous variables will be summarized by descriptive statistics (sample size, mean, standard deviation, median, minimum, and maximum) as well as confidence intervals, where appropriate. Categorical variables will be summarized by frequencies and percentages. Details of the statistical analysis, analysis populations, and data reporting will be provided in the Statistical Analysis Plan (SAP).

### Safety Analyses:

Safety analyses will be presented by cohort and, when appropriate, overall.

Safety assessments, i.e. Vital Signs, ECG, and PE, will be summarized by scheduled visits. Both safety and tolerability will be evaluated by assessing the incidence of DLTs, AEs, ADEs, SAEs and SADEs. All Aes will be coded using the Medical Dictionary for Regulatory Activities (MeDRA).

Laboratory results will be summarized by scheduled visits.

### Feasibility Analyses:

The feasibility analyses will be descriptive in nature. The following endpoints will be summarized by cohort and overall:
The incidence of successful IM and IT injections.
Presence of focal necrosis in bladder tumor (in treated area) assessed using histopathology.
Systemic PK profile and ADA parameters will be summarized.
Within each cohort, the criteria for procedure feasibility is: successful injections in ≥50% of the participants, ≤1 moderate injection-related AE, and ≤1 DLT. Across all cohorts, the overall procedure feasibility criteria is: successful injections in ≥75% of participants, with ≤20% of participants with moderate injection-related Aes, and ≤10% of participants with DLTs.

### Efficacy Analyses:

The efficacy analyses will be descriptive in nature. The following endpoints will be summarized by cohorts and overall:
The 3-month CR rate.
CR at any time.
DOR at the 6-mth, 9-mth and 12-mth timepoint.
Durable CR rate at the 6-mth, 9-mth and 12-mth timepoint.

### Exploratory Analyses:

Participant feedback, blood and urine biomarkers, if collected, will be analyzed in an exploratory manner and will be descriptive in nature. Individual tumor sizes for each participant, as assessed by the Investigators, will be evaluated for any reduction in size over baseline, to potentially determine a lesion-level response in exploratory analyses.

### Sample Size

The trial will enroll up to 55 participants, comprising approximately 34-40 participants with NMIBC in Cohorts 1b and Cohorts 4a-g, and approximately 3 participants with MIBC in the MIBC cohort, with the potential to backfill up to 12 participants. A formal sample size and power calculation have not been performed for the study. This sample size is empiric and based on clinical observation and decision.

Schedule of Pharmacokinetic Sampling for Cohorts 1, 4a-c

| Cohort | D 1/V1 | D 2/V2 | D 5 + 1 D/V3 |
|---|---|---|---|
| Cohort 1b | Pre-injection | Pre-TURBT | Post-op visit |
| | 30 minutes | 30 minutes^{b, c} | Day 5 |
| | Post-injection | Post-TURBT | |
| | 30 minutes | 30 minutesc | |
| | 1 hour | | |
| | 2 hours | | |
| | 24 hours^{b} | | |
| Cohort 4a | Pre-injection | Pre-laser | |
| (50 μg IT and | 30 minutes | 30 minutes | |
| 50 μg IT) | Post- | Post-laser | |
| | injection | 30 minutes | |
| | 30 minutes | 1 hour | |
| | 1 hour | 2 hours | |
| | 2 hours | 24 hours | |
| Cohort 4b and 4c | Pre-injection | Pre-laser | |
| (100 μg IT and | 30 minutes | 30 minutes | |
| 200 μg IT) | Post-injection | Post-laser | |
| | 30 minutes | 30 minutes | |
| | 1 hour | 1 hour | |
| | 2 hours | 2 hours | |

Schedule of Pharmacokinetic Sampling for Cohorts 4d-j and MIBC cohort

| Cohort | V1 | V2 | V3 | V4 | V5 |
|---|---|---|---|---|---|
| 4d, 4e, 4h | 30 min pre- | Before laser | 30 min pre- | Before laser | 3-month f/u |
| | injection | application (~24 | injection | application | visit |
| | 2 hr post- | hr after injection | 2 hr post- | (~24 hr after | |
| | injection | on V1) | injection | injection on V3) | |
| 4g, 4f, 4i | 30 min pre- | Before laser | 30 min pre- | Before laser | TURBT |
| | injection | application (~24 | injection | application | |
| | 2 hr post- | hr after injection | 2 hr post- | (~24 hr after | |
| | injection | on V1) | injection | injection on V3) | |
| 4j | 30 min pre- | 30 min pre- | 30 min pre- | 3-month f/u | |
| | injection | injection | injection | visit | |
| | 30 min before | 30 min before | 30 min before | | |
| | laser application | laser application | laser application | | |
| MIBC | If treatment schedule chosen is that of Cohort 4f, PK | | | | |
| (treatment | sampling will follow schedule for Cohort 4f | | | | |
| schedule | If treatment schedule chosen is that of Cohort 4g, PK | | | | |
| will be | sampling will follow schedule for cohort 4g | | | | |
| determined | If treatment schedule chosen is 2 cycles, same day | | | | |
| by SRB) | injection/laser, PK sampling will be as below: | | | | |
| | 30 min pre- | 30 min pre- | f/u visit at | | |
| | injection | injection | cystectomy | | |
| | 30 min before | 30 min before | | | |
| | laser application | laser application | | | |

^{a}Windows for PK collection are as follows:
30 minutes, 1 hour, and 2 hours (±15 minutes).
24 hours (±1 hour).
Day 5 and Day 9 (±1 day).
^{b}If TURBT performed prior to 24 hours post-injection, collect PK 30 minutes prior to TURBT.
^{c}Window for this visit is Day 2/Visit 2 + 1 day.

### Interim Data Update:

In Cohort 4a-4c, single-dose drug with light activation (n=12)^{a}, fewer than 10% of patients experienced Grade 1 treatment-emergent adverse events (TEAEs) related to the study drug, and no grade 2/3 adverse events related to the study drug (n=17). No serious adverse events or dose-limiting toxicities were observed.

| | | |
|---|---|---|
| Event | Grade | Number of patients |

Adverse events (related to study drμg)

| | | |
|---|---|---|
| Nocturia | 1 | 1/12 |
| Urinary urgency | 1 | 1/12 |

Adverse events (related to injection or laser procedure)

| | | |
|---|---|---|
| Hematuria | 1 | 1/12 |
| Urinary blood clots | 1 | 1/12 |
| Nocturia | 1 | 1/12 |
| Urinary urgency | 1 | 1/12 |
| Dysuria | 1 | 1/12 |

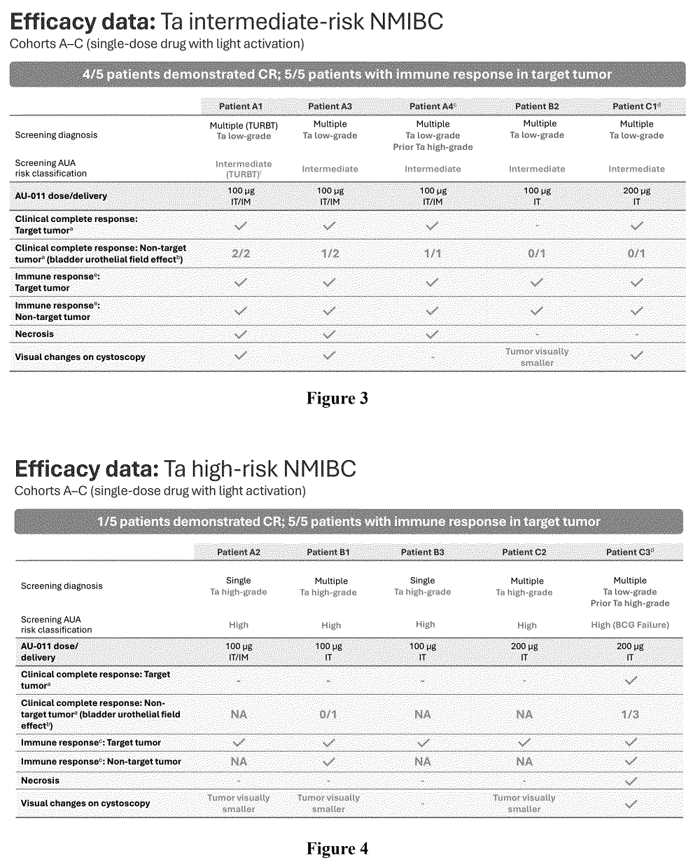

Efficacy data for Ta intermediate-risk NMBIC Cohorts A-C(single-dose drug with light activation) is shown in FIG. 3. Efficacy data for Ta high-risk NMBIC Cohorts A-C(single-dose drug with light activation) is shown in FIG. 4.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. A method for treating a target tumor in a subject in need thereof, the method comprising: injecting into the base of a target tumor and/or into a site proximate to the base of the target tumor a pharmaceutical composition comprising a therapeutic agent in a therapeutically effective amount sufficient to treat the target tumor and induce a therapeutic field effect.

2. A method for treating a target tumor in a subject in need thereof, the method comprising: injecting into the base of a target tumor and/or into a site proximate to the base of the target tumor a pharmaceutical composition comprising a therapeutic agent in a therapeutically effective amount sufficient to transform an immunologically inactive tumor into an immunologically active tumor.

3. The method of claim 1, wherein the therapeutic agent comprises virus-related particles.

4. The method of claim 3, wherein the virus-related particles are selected from viruses, virus-like particles, defective viral particles, and pseudoviruses.

5. The method of claim 3, wherein the therapeutic agent comprises virus-like particles.

6. The method of claim 3, wherein the therapeutic agent comprises a virus-like drug conjugate.

7. The method of claim 1, wherein the therapeutic agent is selected from antibodies and other antigen-binding agents.

8. The method of claim 1, wherein the therapeutic agent is an immunomodulatory agent.

9. The method of claim 1, wherein the pharmaceutical composition further comprises a light-activated moiety.

10. The method of claim 9, wherein the therapeutic agent comprises the light-activated moiety.

11. The method of claim 9, wherein the light-activated moiety comprises a photosensitizer.

12. The method of claim 11, wherein the photosensitizer is selected from porphyrins, aminolevulinic acid, and phthalocyanines.

13. The method of claim 12, wherein the photosensitizer comprises a compound of Formula I or Formula II:

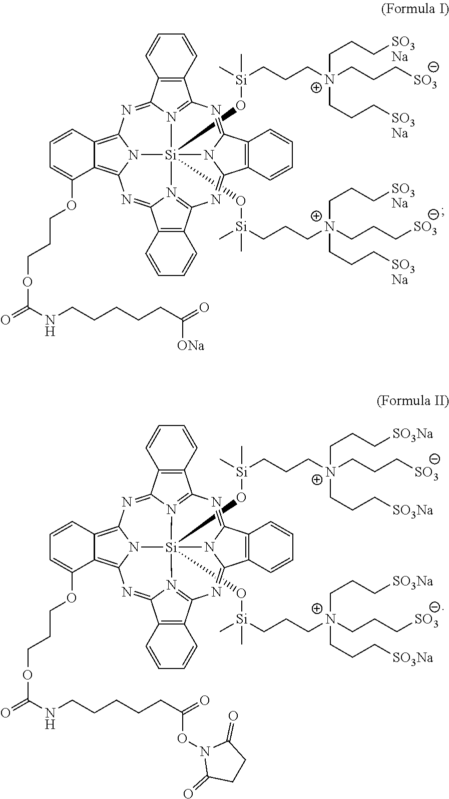

14. (canceled)

15. The method of claim 1, wherein the target tumor is a target urothelial tumor, and the therapeutic field effect is a therapeutic urothelial field effect.

16. The method of claim 15, wherein the target urothelial tumor is a target bladder urothelial tumor, and the therapeutic field effect is a therapeutic bladder urothelial field effect.

17. The method of claim 16, wherein the target bladder urothelial tumor is a non-muscle invasive bladder cancer (NMIBC).

18. The method of any preceding claim, wherein the therapeutic field effect is without systemic exposure to the therapeutic agent and/or is without local or system exposure of an immune checkpoint inhibitor.

19. (canceled)

20. The method of claim 1, wherein the therapeutic field effect is within 7-14 days of injecting the pharmaceutical composition.

21. The method of claim 16, wherein the pharmaceutical composition is delivered below the base of the target tumor and above the muscularis propria.

22. (canceled)

23. The method of claim 9 further comprising exposing the target tumor to a laser.

24.-25. (canceled)

26. The method of claim 1, wherein the therapeutically effective amount is a 200 μg, 400 μg, or 800 μg dose of the therapeutic agent.

27. The method of claim 1, wherein the method comprises administering a single 200 μg, 400 μg, or 800 μg dose of the therapeutic agent via one or more injection into the base of a target tumor and/or into a site proximate to the base of the target tumor, or administering two or more 200 μg, 400 μg, or 800 μg doses of the therapeutic agent doses via two or more injections into the base of a target tumor and/or into a site proximate to the base of the target tumor.

28.-41. (canceled)

42. A method of treating bladder cancer, comprising:
(a) injecting a pharmaceutical composition comprising a photosensitive virus-like drug conjugate (VDC) into one or more bladder cancer lesions of a patient, wherein the pharmaceutical composition is injected at a dose of about 200 μg, about 400 μg, or about 800 μg of the VDC per bladder cancer lesion;
(b) exposing the one or more bladder cancer lesions to a laser treatment at about 24 hours after injecting the pharmaceutical composition into the one or more bladder cancer lesions; and
(c) repeating steps (a) and (b), optionally at least one, two, three, four, or five times.

43. A method for treating bladder cancer, comprising:
(a) performing a first cycle of therapy comprising
(i) injecting a pharmaceutical composition comprising a photosensitive virus-like drug conjugate (VDC) into one or more bladder cancer lesions of a patient, wherein the pharmaceutical composition is injected at a dose of about 200 μg, about 400 μg, or about 800 μg of the VDC per bladder cancer lesion, and
(ii) exposing the one or more bladder cancer lesions to a first laser treatment at about 24 hours after injecting the pharmaceutical composition into the one or more bladder cancer lesions; and
(b) performing a second cycle of therapy comprising
(i) injecting the pharmaceutical composition into the one or more bladder cancer lesions of the patient, wherein the VDC is injected at a dose of about 200 μg, about 400 μg, or about 800 μg per bladder cancer lesion, and
(ii) exposing the one or more bladder cancer lesions to a second laser treatment at about 24 hours after injecting the pharmaceutical composition into the one or more bladder cancer lesions,
wherein the second cycle of therapy is performed about 2 weeks after the first cycle of therapy is performed.

44.-55. (canceled)

56. The method of claim 23, wherein exposing the target tumor to a laser comprises:
(a) advancing a cystoscope comprising a treatment laser into the bladder and positioning the treatment laser over the target tumor midpoint to produce a laser spot size of about 2 cm; and
(b) delivering a total light dose of about 100 J/cm².

57. The method of claim 56, comprising:
(a) advancing into the bladder a cystoscope comprising a working channel into which an open-ended flexitip ureteral catheter has been inserted;
(b) positioning the cystoscope over the tumor midpoint at a distance of about 4 cm from the bladder wall;
(c) removing the catheter without moving the cystoscope;
(d) inserting a light delivery catheter comprising a 1 cm tip into the working channel of the cystoscope such that the 1 cm tip is positioned outside the cystoscope about 3 cm away from the bladder wall to produce a 2 cm laser spot size, wherein the light delivery catheter comprises an aiming laser and a treatment laser;
(e) targeting light at the target tumor using the aiming laser to set the target area; and
(f) activating the treatment laser within the target area to deliver a total light dose of about 100 J/cm².

58.-59. (canceled)
